(19) United States
(12) Patent Application Publication
(10) Pub. No.: US 2023/0304099 A1
(43) Pub. Date: Sep. 28, 2023

(54) Gene Expression Signature for Predicting Immunotherapy Response and Methods of Use

(71) Applicant: The Medical College of Wisconsin, Inc., Milwaukee, WI (US)

(72) Inventors: Donghai Xiong, Milwaukee, WI (US); Ming You, Milwaukee, WI (US); Yian Wang, Milwaukee, WI (US)

(21) Appl. No.: 18/041,534

(22) PCT Filed: Aug. 9, 2021
(86) PCT No.: PCT/US2021/045191

§ 371 Date: Feb. 13, 2023

Related U.S. Application Data
(60) Provisional application No. 63/066,079, filed on Aug. 14, 2020.

(51) Int. Cl. C12Q 1/6886; G16B 40/00

(52) U.S. Cl. CPC C12Q 1/6886; G16B 40/00; C12Q 2600/106; C12Q 2600/158

(57) ABSTRACT

The present invention provides novel gene signature associated with immune checkpoint inhibitor (ICT) named ImmuneCells.Sig which is predicative of ICT outcomes of melanoma patients which is significantly more accurate than all previously reported ICT response signatures. The ImmuneCells.Sig can be used as an accurate predictor of ICT response and may be used to determine if a patient will be susceptible and respond to ICT treatment.

### CROSS-REFERENCE TO RELATED APPLICATIONS

This application claims the benefit of priority under 35 U.S.C. § 119(e) of U.S. Provisional Application No. 63/066,079, filed Aug. 14, 2020, the contents of which is incorporated herein by reference in its entirety.

### STATEMENT REGARDING FEDERALLY SPONSORED RESEARCH

This invention was made with government support under R01 CA134682 awarded by the National Institute of Health. The Government has certain rights in the invention.

### BACKGROUND OF THE INVENTION

While immune checkpoint therapies (ICT) have improved outcomes for some cancer patients, most patients do not respond to ICT. Identifying factors underlying resistance to immune checkpoint therapy (ICT) is still challenging. Previous whole-exome sequencing (WES) and transcriptome sequencing of tumors identified multiple factors that are associated with favorable ICT outcome, including expression of PD-L1¹², high tumor mutational burden¹³, and the presence of tumor-infiltrating CD8+ T cells¹⁴. Markers indicative of unfavorable response include defects in IFNγ pathways or antigen presentation^{15,16}. However, previous efforts to discover biomarkers for patients who will respond to ICT mainly focused on CD8+ T cells¹⁷.

### SUMMARY OF THE INVENTION

The present invention addresses the aforementioned need by determining if other types of immune cells and their subclusters are associated with ICT outcomes. While prior studies represented a first step in identifying biomarkers, studies using single-cell RNA sequencing (scRNA-seq) have the potential to greatly improve the identification of factors underlying ICT outcomes. The inventors identified a previously unrecognized immune cell subpopulations that could play an important role in determining ICT responsiveness. The analysis of multiple additional gene expression datasets of more melanoma samples identified and validated an ICT outcome signature (“ImmuneCells.Sig”) enriched with the genes characteristic of the immune cell subsets detected in the scRNA-seq study. It predicted the ICT outcomes of melanoma patients more accurately than the 12 previously reported ICT response signatures. The validated ImmuneCells.Sig provided an improved predictor of ICT response and could contribute to the decision making for immunotherapy, particularly anti-PD-1 therapy.

The present disclosure thereby provides a novel gene expression signature (ImmuneCells.Sig) that predicted the ICT (immune checkpoint therapy) outcomes of melanoma patients with significantly more accuracy than all previously reported ICT response signatures. The validated ImmuneCells.Sig provided one of the most accurate predictors to date of ICT response and could contribute immensely to clinical decision making for immunotherapy. The gene expression signature may be provided in a chip or detection kit for determining ICT responsiveness of a tumor.

The present invention provides methods and kits for determining if a subject would be susceptible to immune checkpoint therapy, the method comprising detecting one or more genes associated with the gene expression signature as described in the Examples section. In some embodiments, the one or more genes is detected by RNA sequencing (RNA-seq). In some embodiments, the one or more genes is detected by single-cell RNA sequencing (scRNA-seq).

In one aspect, the present invention provides a method of determining susceptibility and response to immune checkpoint therapy in a subject in need thereof, the method comprising detecting one or more genes associated with an immune cell gene expression signature (ImmuneCells.Sig) of Table 1, wherein the detecting of one or more of the genes detects resistance to the immune checkpoint therapy.

In another aspect, the invention provides a method of treating a subject with cancer, the method comprising: a) determining if the subject has a cancer which is susceptible and responsive to a checkpoint inhibitor by determining expression profile of one or more genes associated with an immune cell gene expression signature (ImmuneCells.Sig), and b) treating the subject with the checkpoint inhibitor in an amount effective to treat the cancer.

In a further aspect, the disclosure provides a gene chip comprising an expression signature (ImmuneCells.Sig) useful for determining the response to immune checkpoint therapy, the gene chip comprising probes useful to detect the level of 10 or more biomarkers listed in Table 1.

In yet another aspect, the disclosure provides method for processing a test sample to determine a likelihood that a cancer is responsive to anti-PD-1 immunotherapy in a patient, comprising: (a) receiving information indicative of an expression level of a plurality of biomarkers in a tumor sample extracted from the patient; (b) providing the plurality of biomarker levels as input to a classifier configured to predict likelihood that a patient is reactive in response to anti-PD-1 immunotherapy in a computer to classify the test sample, wherein the classifier was trained with a plurality of training samples comprising pre-therapy tumor expression data of known PD-1 therapy responding patients and pre-therapy tumor expression data of known non-responder patients, and wherein the sensitivity and specificity of the classifier is sufficient to identify the likelihood that the patient is responsive to anti-PD-1 immunotherapy; (c) receiving, from the classifier, an output report that identifies said classification as indicative of the likelihood that the patient is responsive to anti-PD-1 immunotherapy.

In yet another aspect, the invention provides a kit for detecting the likelihood of a subject with cancer to be responsive to checkpoint therapy, the kit comprising a panel of 10 biomarkers from Table 2 attached to a solid surface and instructions for use.

In a further aspect, the invention provides a system for processing a test sample to determine a likelihood that a patient with cancer is responsive to anti-PD-1 immunotherapy in a patient, comprising: (a) a computer capable of receiving input data of the expression of a plurality of biomarker levels, (b) a classifier configured to predict likelihood that a to respond to anti-PD-1 immunotherapy to classify the test sample, and (c) an output report from the classifier that identifies said classification as indicative of the likelihood that the patient be responsive to anti-PD-1 immunotherapy.

The foregoing and other aspects and advantages of the invention will appear from the following description. In the description, reference is made to the accompanying drawings which form a part hereof, and in which there are shown, by way of illustration, preferred embodiments of the invention. Such embodiments do not necessarily represent the full scope of the invention, however, and reference is made therefore to the claims and herein for interpreting the scope of the invention.

### BRIEF DESCRIPTION OF THE DRAWINGS

FIG. 1A-1D. Identification of intratumoral immune cell populations by scRNA-seq. The scRNA-seq dataset—GSE120575 was analyzed. (a) Uniform manifold approximation and projection (UMAP) plot of intratumoral immune cells that were classified into 23 clusters from the two groups of melanoma samples of distinct immune checkpoint therapy outcomes (NR [non-responder] group versus R [responder] group). (b) UMAP plot of the 10 major immune cell populations. (c) Comparison of the cell cluster percentage changes between the NR and R groups. Boxplots showed the results for the nine immune cell clusters with significant changes. Center line, median. Box limits, upper and lower quartiles. Whiskers, 1.5 interquartile range. Points beyond whiskers, outliers. The two-sided Wilcoxon tests were performed with no adjustment for multiple comparisons. (d) The fold changes of the percentages of each of the 23 single-cell clusters comparing the NR group to the R group.

FIG. 2A-2B. Subsets of macrophages in the melanoma tumors. The scRNA-seq dataset—GSE120575 was used in this analysis. (a) Heatmap of z-scored expression of the top up-regulated genes of each macrophage subpopulation versus the other two macrophage subpopulations. (b) Violin plots of log-transformed gene expression of selected genes showing statistically significant upregulation in inflammatory macrophages (top), TREM2^{hi} macrophages (center), and Immunoregulatory related macrophages (bottom).

FIG. 3A-3C. The analysis of the gammadelta T cells (Tgd) cells and B cells subsets in the melanoma samples. The scRNA-seq dataset—GSE120575 was used in this analysis. (a) Heatmap of z-scored expression of the top up-regulated genes of the Tgd subpopulations—Tgd_c8 and Tgd_c21. (b) Heatmap of z-scored expression of the top up-regulated genes of the B-cells subpopulations—B_c13, B_c14, B_c17, and B_c22. (c) The significantly altered molecular pathways in the Tgd_c21 and B_c22 immune cell subpopulations whose percentages were associated with ICT outcomes.

FIG. 4A-4D. The ImmuneCells.Sig signature may predict ICT outcome in melanoma patients. (a) ImmuneCells.Sig had significantly high prognostic values for ICT outcomes in the initial discovery dataset—GSE78220. (b) ImmuneCells.Sig accurately predicted the ICT outcome in the first validation dataset of GSE91061. (c) ImmuneCells.Sig accurately predicted the ICT outcome in the second validation dataset of PRJEB23709. (d) ImmuneCells.Sig accurately predicted the ICT outcome in the third validation dataset of MGSP.

FIG. 5A-5D. Comparison of the performance of ImmuneCells.Sig with other ICT response signatures. The multiple barplots for the AUC values of the 13 ICT response signatures are shown in (a) for the GSE78220 dataset. (b) for the GSE91061 dataset. (c) for the PRJEB23709 dataset. (d) for the MGSP dataset.

FIG. 6. Table 4. The list of biomarkers for response to immune checkpoint therapy that were compared in this disclosure.

FIG. 7. The characterization of the ten major immune cell populations according to their respective canonical marker expression status. The scRNA-seq dataset—GSE120575 was used in this analysis. (a) The expression of canonical marker genes in different clusters of single cells; (b) Validation of our defined gd T lymphocytes by the expression of the published gene expression signatures of gd T cells.

FIG. 8. Comparison of the abundance of each immune cell subset between the immune checkpoint therapy responder and non-responder groups. The scRNA-seq dataset—GSE120575 was used in this analysis. Boxplots showing the results of the Wilcoxon tests at the patient level for each of the 23 immune cell clusters were presented. There were 17 samples for the responder group and 31 samples for the non-responder group. Center line, median. Box limits, upper and lower quartiles. Whiskers, 1.5 interquartile range. Points beyond whiskers, outliers. The two-sided Wilcoxon tests were performed with no adjustment for multiple comparisons.

FIG. 9A-9C. Cell abundance comparison stratified by treatment schemes. The scRNA-seq dataset—GSE120575 was used in this analysis. The percentages (% of CD45+ cells) of each of the 23 single-cell clusters for the responders (R) and non-responders (NR) groups of melanoma samples collected in the three scenarios. (a) before anti-PD-1 treatment; (b) after anti-PD-1 treatment; (c) after anti-CTLA4+ anti-PD-1 treatment. No enough single cells were available for comparison between responders and non-responders for other scenarios. For the after anti-PD-1 treatment melanoma samples, the denominators for the R and the NR groups are 1524 and 6334, respectively; for the after anti-CTLA4 plus anti-PD-1 treatment melanoma samples, the denominators for the R and the NR groups are 1315 and 1190, respectively.

FIG. 10. Fraction of each macrophage subsets. The scRNA-seq dataset—GSE120575 was used in this analysis. Proportions of inflammatory macrophages (cluster 6), TREM2^{hi} macrophages (cluster 12), and Immunoregulatory related macrophages (cluster 23), the three macrophage subsets in immune cells from the melanoma tumor samples.

FIG. 11. Pathway analysis for macrophage cluster 6. The scRNA-seq dataset—GSE120575 was used in this analysis. IPA analyses based on the list of differentially expressed genes between cluster 6 and other macrophages. The results revealed that inflammatory response was significantly activated with a large number of overexpressed inflammatory marker genes in cluster 6 macrophages (adjusted P=3.93E−10, activation Z score=2.01). Cluster 6 macrophages population was thus identified as the ‘Inflammatory Mf’.

FIG. 12A-12C. Gene ontology enrichment analysis of three macrophages subsets. The scRNA-seq dataset—GSE was used in this analysis. Gene ontology enrichment analysis of reactome pathways in (a) Inflammatory macrophages, (b) TREM2^{hi} macrophages, and (c) Immunoregulatory related macrophages infiltrating the melanoma tumor samples from patients subjected to ICT. Size of the circles is proportional to the fold difference.

FIG. 13A-13D. A 40-gene expression signature that can characterize the TREM2^{hi} macrophage population. (a) The scRNA-seq dataset—GSE120575 was analyzed, which generated the heatmap of the expression of a 40-gene signature representing the TREM2^{hi} macrophage population. In the boxplots for (b) GSE78220 dataset and (c) GSE91061 dataset, the GSVA scores of the TREM2^{hi} macrophage geneset were significantly higher in the ICT non-responding tumors than the responding tumors. Center line, median. Box limits, upper and lower quartiles. Whiskers, 1.5 interquartile range. Points beyond whiskers, outliers. For (b) and (c), the two-sided t-tests were performed with no adjustment for multiple comparisons. (d) Violin plot showed that the activity of this gene set is higher in the TREM2^{hi} macrophages compared to the other macrophages in the GSE120575 dataset.

FIG. 14A-14F. Validation of the findings in melanoma using an independent scRNAseq dataset of melanoma—GSE115978. (a)-(b) The deeper clustering of the macrophages and B cells sequenced by Jerby-Arnon et al.'s scRNA-seq study showed the existence of the similar macrophage and B cell subpopulations that resemble our identified TREM2^{hi} macrophages and B_c22 B cells; (c) Heatmap of the four macrophage subclusters, which showed that the ‘Mac_c1a’ macrophage subcluster overexpressed the TREM2hi macrophage marker genes; (d) Heatmap of the three B cells subclusters, which showed that the ‘B_s1’ B cell subcluster overexpressed the B_c22 B cells marker genes; (e) the Mac_c1 macrophage subset had significantly higher overall expression of the TREM2^{hi} macrophage signature in the non-responders than the control samples; (f) The B_s1 B cell subset had significantly lower overall expression of the B_c22 B cell signature in the immunotherapy non-responders than the control samples. Center line, median. Box limits, upper and lower quartiles. Whiskers, 1.5 interquartile range. Points beyond whiskers, outliers. For (e) and (f), the two-sided Wilcoxon tests were performed with no adjustment for multiple comparisons.

FIG. 15A-15F. Validation of the findings in melanoma using an scRNAseq dataset of basal cell carcinoma (BCC) —GSE123813. (a) General clustering analyses identified the overall macrophages and B cells populations; Finer clustering identified the macrophage (b) and B cell subpopulations (c) from the BCC tumors that are similar to the TREM2^{hi} macrophages and B_c22 B cells in the initial melanoma samples; (d) Heatmap showed that in the BCC dataset the ‘Mac_s2’ macrophage subcluster overexpressed the TREM2hi macrophage marker genes; (e) Heatmap showed that ‘B_sc2’ B cell subcluster overexpressed the B_c22 B cells marker genes; (f) The Mac_s2 macrophage subset had significantly decreased overall expression of the TREM2^{hi} macrophage signature in the responsive BCC tumors after the anti-PD-1 therapy than the pretreatment BCC samples; (g) The B_sc2 B cell subset had significantly higher overall expression of the B_c22 signature in the post anti-PD-1 therapy responsive BCC tumors than the pretreatment BCC samples. Center line, median. Box limits, upper and lower quartiles. Whiskers, 1.5 interquartile range. Points beyond whiskers, outliers. For (f) and (g), the two-sided Wilcoxon tests were performed with no adjustment for multiple comparisons.

FIG. 16A-16C. The enrichment of the ImmuneCells.Sig signature for the characteristic genes of the immune cell subpopulations. The dataset—GSE78220 was used in this analysis. This ICT outcome signature was positively enriched for the characteristic genes of the (a) TREM2hi Mf, (b) Tgd_c21, and negatively enriched for the (c) B_c22.

FIG. 17A-17B. Evaluation of the ImmuneCells.Sig signature. The dataset—PRJEB23709 was used in this analysis. The performance of the ImmuneCells.Sig signature in predicting ICT responders based on the pre-treatment melanoma biopsies from patients subjected to different ICT regimen. ImmuneCells.Sig can accurately distinguish responders from non-responders in both Pre_anti-PD-1 and Pre_Combo subgroups (anti-PD-1 plus anti-CTLA-4) as can be seen in the ROC (receiver operating characteristic) curves of the (a) PRJEB23709_Pre_anti-PD-1 subset and (b) PRJEB23709_Pre_Combo subset.

FIG. 18A-18D. Comparison of the performance of ImmuneCells.Sig with other ICT response signatures. The multiple ROC (receiver operating characteristic) curves are shown for the 13 ICT response signatures in (a) for the GSE78220 dataset; (b) for the GSE91061 dataset; (c) for the PRJEB23709 dataset and (d) for the MGSP dataset.

FIG. 19. The expression of M1 macrophage marker genes in the TREM2^{hi} population cells. The scRNA-seq dataset—GSE120575 was used in this analysis.

FIG. 20A-20D. Performance of a signature of immune cells. Using the gene signature of the three component cell clusters identified from single-cell data (TREM2^{hi} macrophages, Tgd_c21 γδ T cells and B_c22 B cells) to perform ICT outcome prediction analyses. The AUC values from this signature were 0.92, 0.90, 0.84 and 0.78 for the datasets of (a) GSE78220, (b) GSE91061, (c) PRJEB23709, and (d) MGSP, respectively.

FIG. 21. Table 5. The percentages of the cluster-specific cells of the CD45+ immune cells for each group (Responder—‘R’; Non-Responder—‘NR’). The scRNA-seq dataset—GSE120575 was used in this analysis.

FIG. 22. Table 6. Melanoma sample number and single cell number of stratified groups. The scRNA-seq dataset—GSE120575 was used in this analysis.

FIG. 23 is a diagram depict RNA-seq for providing the input data.

FIG. 24 is a diagram depicting the system of the current invention.

### DETAILED DESCRIPTION OF THE INVENTION

Despite progress in the development of immune checkpoint therapies (ICT), identifying factors underlying ICT resistance is still challenging. Most cancer patients do not respond to ICT and the availability of the predictive biomarkers is limited. Here, we analyzed a single-cell RNA sequencing (scRNA-seq) dataset of tumor-infiltrating immune cells from 48 melanoma samples of patients subjected to ICT and discovered a subset of macrophages (cluster 12) overexpressing TREM2 and a subset of γδ T cells (cluster 21) that were both overrepresented in the non-responding tumors. In addition, the percentage of a B cell subset (cluster 22) was significantly lower in the non-responders. The presence of the immune cell subtypes was corroborated in other scRNA-seq datasets including that of another cancer type. The analysis of multiple gene expression datasets of the melanoma samples identified and validated an ICT outcome signature—ImmuneCells.Sig enriched with the genes characteristic of the above immune cell subsets. ImmuneCells.Sig predicted the ICT outcomes of melanoma patients with significantly more accuracy than all previously reported ICT response signatures. The validated ImmuneCells.Sig provided one of the most accurate predictors to date of ICT response and could contribute immensely to clinical decision making for immunotherapy.

The present invention provides novel gene signature associated with immune checkpoint inhibitor (ICT) named ImmuneCells.Sig which is predicative of ICT outcomes of cancer patients, e.g., melanoma patients, which is significantly more accurate than all previously reported ICT response signatures. The ImmuneCells.Sig can be used as an accurate predictor of ICT response and may be used to determine if a patient will be susceptible and respond to ICT treatment.

The methods and compositions of the current disclosure pertain to signatures used to determine if a patient will be susceptible and respond to ICT treatment.

As used herein, “immune checkpoints” refers to proteins or peptides that regulate the activity of an immune response. For example, some immune checkpoints interfere with the ability of the immune system to mount an effective response. By way of example but not by way of limitation, immune checkpoints include the PD-1:PD-L1/PD-L2 axis.

As used herein, “immune checkpoint therapy” (“ICT”) refers to an intervention that is targeted to interfere with the normal function of “immune checkpoints.” In some embodiments, ICT comprises a treatment that interferes with the function of PD-1 or its ligands PD-L1 and PD-L2. In some embodiments, the ICT comprises a monoclonal antibody targeted to PD-1. In some embodiments, the monoclonal ICT therapy is selected from the group consisting of pembrolizumab, nivolumab, cemiplimab, atezolizumab, dostarlimab, durvalumab, and avelumab.

Checkpoint inhibitors that comprise anti-PD1 antibodies or anti-PDL1-antibodies or fragments thereof are known to those skilled in the art, and include, but are not limited to, cemiplimab, nivolumab, pembrolizumab, MEDI0680 (AMP-514), spartalizumab, camrelizumab, sintilimab, toripalimab, dostarlimab, and AMP-224. Checkpoint inhibitors that comprise anti-PD-L1 antibodies known to those skilled in the art include, but are not limited to, atezolizumab, avelumab, durvalumab, and KN035. The antibody may comprise a monoclonal antibody (mAb), chimeric antibody, antibody fragment, single chain, or other antibody variant construct, as known to those skilled in the art. PD-1 inhibitors may include, but are not limited to, for example, PD-1 and PD-L1 antibodies or fragments thereof, including, nivolumab, an anti-PD-1 antibody, available from Bristol-Myers Squibb Co and described in U.S. Pat. Nos. 7,595,048, 8,728,474, 9,073,994, 9,067,999, 8,008,449 and 8,779,105; pembrolizumab, and anti-PD-1 antibody, available from Merck and Co and described in U.S. Pat. Nos. 8,952,136, 83,545,509, 8,900,587 and EP2170959; atezolizumab is an anti-PD-L1 available from Genentech, Inc. (Roche) and described in U.S. Pat. No. 8,217,149; avelumab (Bavencio, Pfizer, formulation described in PCT Publ. WO2017097407), durvalumab (Imfinzi, Medimmune/AstraZeneca, WO2011066389), cemiplimab (Libtayo, Regeneron Pharmaceuticals Inc., Sanofi, see, e.g., U.S. Pat. Nos. 9,938,345 and 9,987,500), spartalizumab (PDR001, Novartis), camrelizumab (AiRuiKa, Hengrui Medicine Co.), sintilimab (Tyvyt, Innovent Biologics/Eli Lilly), KN035 (Envafolimab, Tracon Pharmaceuticals, see, e.g., WO2017020801A1); tislelizumab available from BeiGene and described in U.S. Pat. No. 8,735,553; among others and the like. Other PD-1 and PD-L1 antibodies that are in development may also be used in the practice of the present invention, including, for example, PD-1 inhibitors including toripalimab (JS-001, Shanghai Junshi Biosciences), dostarlimab (GlaxoSmithKline), INCMGA00012 (Incyte, MarcoGenics), AMP-224 (AstraZeneca/MedImmune and GlaxoSmithKline), AMP-514 (AstraZeneca), and PD-L1 inhibitors including AUNP12 (Aurigene and Laboratoires), CA-170 (Aurigen/Curis), and BMS-986189 (Bristol-Myers Squibb), among others (the references citations regarding the antibodies noted above are incorporated by reference in their entireties with respect to the antibodies, their structure and sequences). Fragments of PD-1 or PD-L1 antibodies include those fragments of the antibodies that retain their function in binding PD-1 or PD-L1 as known in the art, for example, as described in AU2008266951 and Nigam et al. “Development of high affinity engineered antibody fragments targeting PD-L1 for immunoPED,” J Nucl Med May 1, 2018 vol. 59 no. supplement 1 1101, the contents of which are incorporated by reference in their entireties.

As used herein, “cancer” refers to many diseases, e.g., cell proliferative diseases, wherein an organism's cells grow uncontrollably and may spread to other locations in the organism. By way of example but not by way of limitation, cancer may refer to breast cancer, lung cancer, prostate cancer, skin cancer, colon cancer, leukemia, or lymphoma. In some embodiments, cancer refers to melanoma. In some embodiments, cancer refers to basal cell carcinoma (BSC). Specifically, the cancers may be cancers in which checkpoint inhibitors are used for treatment, including anti-PD-1 therapies.

In a first aspect of the current disclosure, methods of determining susceptibility and response to immune checkpoint therapy in a subject in need thereof are provided. As used herein, “susceptibility” refers to the expectation that a patient will respond positively to the indicated therapy. As used herein, “response” refers to a condition where therapeutic targets, for example, tumor burden, that have been defined a priori have been significantly modified by treatment. Modification of treatment includes a reduction of tumor burden, inhibition or reduction of tumor growth and the like. In some embodiments, the method comprises detecting one or more genes associated with an immune cell gene expression signature (ImmuneCells.Sig) of Table 1, wherein the detecting of one or more of the genes detects resistance to the immune checkpoint therapy. In some embodiments of the method, the subject has melanoma. In some embodiments, the subject has basal cell carcinoma (BCC). In some embodiments, the method comprises treating the subject with immune checkpoint therapy if the one or more genes is not detected. In some embodiments, the one or more genes are associated with macrophages that overexpress TREM2 or a subset of γδ T cells. In some embodiments, the one or more genes comprises 10, 20, 30, 40, 50, 60, 70, 80, 90, 100, or 108 of the biomarkers listed in Table 1.

In a second aspect of the current disclosure, methods of treating a subject with cancer are provided. In some embodiments, the method comprises: a) determining if the subject has a cancer which is susceptible and responsive to a checkpoint inhibitor by determining expression profile of one or more genes associated with an immune cell gene expression signature (ImmuneCells.Sig), and b) treating the subject with the checkpoint inhibitor in an amount effective to treat the cancer. In some embodiments, the cancer is melanoma. In some embodiments, the checkpoint inhibitor is PD-1 or PD-L1 inhibitor. In some embodiments, the one or more genes comprise 10, 20, 30, 40, 50, 60, 70, 80, 90, 100, or 108 of the biomarkers listed in Table 1.

In a third aspect of the current disclosure, a gene chip is provided. In some embodiments, the gene chip comprises an expression signature (ImmuneCells.Sig) useful for determining the response to immune checkpoint therapy, the gene chip comprising probes useful to detect the level of 10 or more biomarkers listed in Table 1. In some embodiments, the gene chip comprises probes useful to detect the level 20, 30, 40, 50, 60, 70, 80, 90, 100, or 108 of the biomarkers listed in Table 1. In some embodiments, the chip comprises 108 biomarkers listed in Table 1.

In a fourth aspect of the current disclosure, methods for processing a test sample to determine a likelihood that a cancer is responsive to anti-PD-1 immunotherapy in a patient are provided. In some embodiments, the methods comprise (a) receiving information indicative of an expression level of a plurality of biomarkers in a tumor sample extracted from the patient; (b) providing the plurality of biomarker levels as input to a classifier configured to predict likelihood that a patient is reactive in response to checkpoint therapy, preferably anti-PD-1 immunotherapy, in a computer to classify the test sample, wherein the classifier was trained with a plurality of training samples comprising pre-therapy tumor expression data of known PD-1 therapy responding patients and pre-therapy tumor expression data of known non-responder patients, and wherein the sensitivity and specificity of the classifier is sufficient to identify the likelihood that the patient is responsive to anti-PD-1 immunotherapy; (c) receiving, from the classifier, an output report that identifies said classification as indicative of the likelihood that the patient is responsive to anti-PD-1 immunotherapy. In some embodiments, the method for processing a test sample further comprises: determining, based on the output, that the patient is likely responsive to anti-PD-1 immunotherapy; and administering anti-PD-1 immunotherapy to the patient based on the determination that the patient is likely to respond to anti-PD-1 immunotherapy.

As used herein, “biomarker” refers to a biological molecule found in blood, other body fluids, or tissues that is a sign of a normal or abnormal process, or of a condition or disease. A biomarker may be used to see how well the body responds to a treatment for a disease or condition. Also called molecular marker and signature molecule.

In some embodiments, the classifier has an accuracy of at least 85%. In some embodiments, the method comprises: detecting the expression level of the plurality of biomarkers by sequencing the nucleic acid molecules from the sample to yield data comprising one or more levels of gene expression producing is the sample. In some embodiments, the method comprises RNA sequencing (RNA-seq) analysis. As used herein, “sequencing” refers to the sequencing of nucleic acids. Sequencing of nucleic acids may be accomplished using, by way of example but not by way of limitation, Sanger sequencing, or next-generation sequencing. In some embodiments, the plurality of biomarkers comprises 10, 20, 30, 40, 50, 60, 70, 80, 90, 100, or 108 of the biomarkers listed in Table 1. In some embodiments, the plurality of biomarkers consists of the 10 biomarkers in Table 2. In some embodiments, the patient's tumor is of a type selected from the group consisting of melanoma and basal cell carcinoma (BCC). In some embodiments of the methods, step (b) comprises identifying a copy number variation or a variant in the nucleotide data. In some embodiments of the method, said known samples comprise a cancer tissue sample from melanoma or basal cell carcinoma (BCC). In some embodiments, said plurality of training samples further comprises a normal tissue sample. In some embodiments of the method, said sensitivity is at least 70%. In some embodiments, said classifier generates said classification at a specificity of at least about 90%, alternatively at least 95%. In some embodiments, said sample of melanoma tissue was from a patient that was sensitive to checkpoint inhibitor therapy, preferably anti-PD-1 therapy, and wherein said classifier does classify said sample as likely to be responsive to the checkpoint inhibitor therapy. In some embodiments, said sample of melanoma tissue was from a patient treated with anti-PD therapy that was not responsive to checkpoint therapy, and wherein said classifier classifies said sample of melanoma tissue as not likely to be responsive to checkpoint therapy. In some embodiments, the method further comprises providing a treatment to said subject.

In a fifth aspect of the current disclosure, a kit for detecting the likelihood of a subject with cancer to be responsive to checkpoint therapy is provided. In some embodiments, the kit comprises a panel of 10 biomarkers from Table 2 attached to a solid surface and instructions for use.

In a sixth aspect of the current disclosure, systems for processing a test sample to determine a likelihood that a patient with cancer is responsive to anti-PD-1 immunotherapy in a patient are provided. In some embodiments, the system comprises: (a) a computer capable of receiving input data of the expression of a plurality of biomarker levels, (b) a classifier configured to predict likelihood that a to respond to anti-PD-1 immunotherapy to classify the test sample, and (c) an output report from the classifier that identifies said classification as indicative of the likelihood that the patient be responsive to anti-PD-1 immunotherapy.

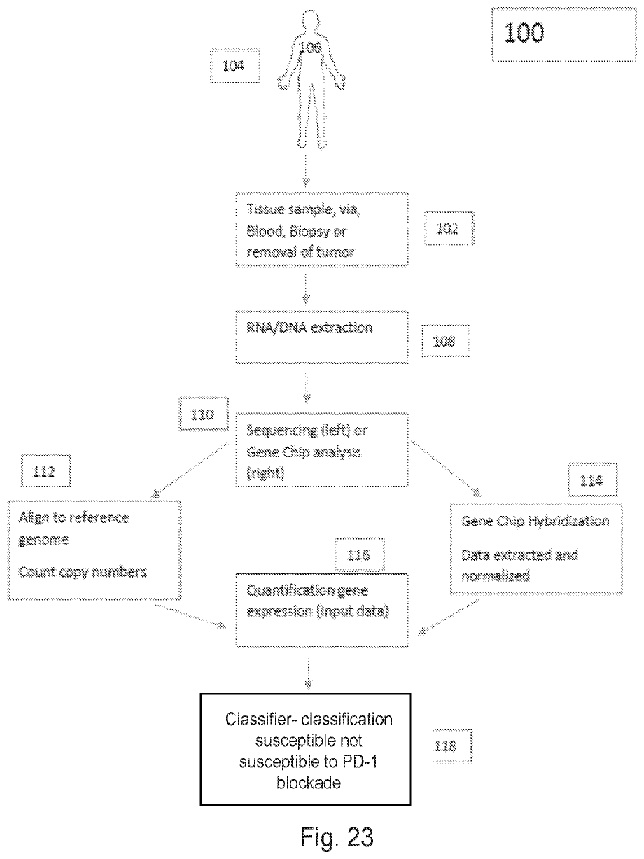

The present disclosure provides systems that are programmed to implement methods of the disclosure. FIG. 23 shows a computer system 100 that is programmed or otherwise configured to classify individuals as susceptible or not susceptible to PD-1 therapy (118). This determination, analysis or statistical classification is done by methods known in the art, including, but not limited to, for example, a wide variety of supervised and unsupervised data analysis and clustering approaches such as hierarchical cluster analysis (HCA), principal component analysis (PCA), Partial least squares Discriminant Analysis (PLS-DA), machine learning (also known as random forest), logistic regression, decision trees, support vector machine (SVM), k-nearest neighbors, naive bayes, linear regression, polynomial regression, SVM for regression, K-means clustering, and hidden Markov models, among others. The computer system 200 can perform various aspects of analyzing the gene expression data (input data) of the present disclosure, such as, for example, comparing/analyzing the disease state. The computer system can be used for running the classifiers to detect and discriminate different disease states (e.g., responsive to anti-PD1 therapy vs. non-responsive to PD-1 therapy). Data collected can be used to train a machine learning algorithm, specifically an algorithm that receives array measurements from a patient. Before training the algorithm, raw data from the array can be first de-noised to reduce variability in individual variables.

Generally, machine learning algorithms are used to construct models that accurately assign class labels to examples based on the input features that describe the example. In some case it may be advantageous to employ machine learning and/or deep learning approaches for the methods described herein. Further, machine learning can be understood as the ability of a learning machine to perform accurately on new, unseen examples/tasks after having experienced a learning data set. Machine learning may include the following concepts and methods. Supervised learning concepts may include AODE; Artificial neural network, such as Backpropagation, Autoencoders, Hopfield networks, Boltzmann machines, Restricted Boltzmann Machines, and Spiking neural networks; Bayesian statistics, such as Bayesian network and Bayesian knowledge base; Case-based reasoning; Gaussian process regression; Gene expression programming; Group method of data handling (GMDH); Inductive logic programming; Instance-based learning; Lazy learning; Learning Automata; Learning Vector Quantization; Logistic Model Tree; Minimum message length (decision trees, decision graphs, etc.), such as Nearest Neighbor Algorithm and Analogical modeling; Probably approximately correct learning (PAC) learning; Ripple down rules, a knowledge acquisition methodology; Symbolic machine learning algorithms; Support vector machines; Random Forests; Ensembles of classifiers, such as Bootstrap aggregating (bagging) and Boosting (meta-algorithm); Ordinal classification; Information fuzzy networks (IFN); Conditional Random Field; ANOVA; Linear classifiers, such as Fisher's linear discriminant, Linear regression, Logistic regression, Multinomial logistic regression, Naive Bayes classifier, Perceptron, Support vector machines; Quadratic classifiers; k-nearest neighbor; Boosting; Decision trees, such as C4.5, Random forests, ID3, CART, SLIQ, SPRINT; Bayesian networks, such as Naive Bayes; and Hidden Markov models. Unsupervised learning concepts may include; Expectation-maximization algorithm; Vector Quantization; Generative topographic map; Information bottleneck method; Artificial neural network, such as Self-organizing map; Association rule learning, such as, Apriori algorithm, Eclat algorithm, and FP-growth algorithm; Hierarchical clustering, such as Single-linkage clustering and Conceptual clustering; Cluster analysis, such as, K-means algorithm, Fuzzy clustering, DBSCAN, and OPTICS algorithm; and Outlier Detection, such as Local Outlier Factor. Semi-supervised learning concepts may include; Generative models; Low-density separation; Graph-based methods; and Co-training. Reinforcement learning concepts may include; Temporal difference learning; Q-learning; Learning Automata; and SARSA. Deep learning concepts may include; Deep belief networks; Deep Boltzmann machines; Deep Convolutional neural networks; Deep Recurrent neural networks; and Hierarchical temporal memory.

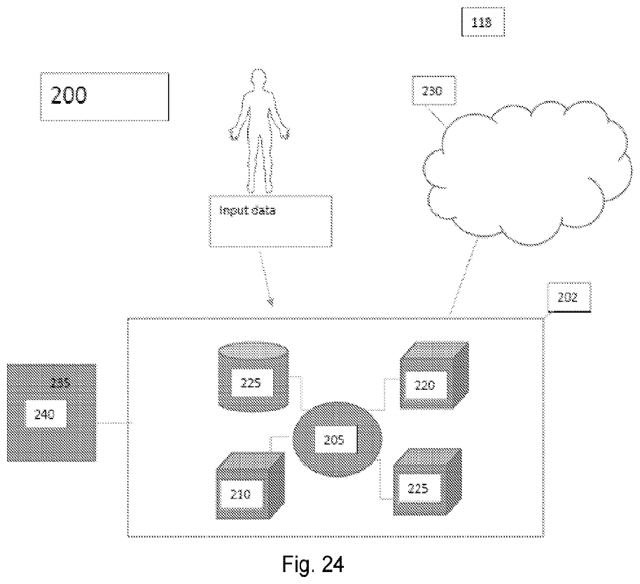

The computer system 200 depicted in FIG. 24 is adapted to implement a method described herein. The system 200 includes a central computer server 202 that is programmed to implement exemplary methods described herein. The server 202 includes a central processing unit (CPU, also “processor”) 204 which can be a single core processor, a multi core processor, or plurality of processors for parallel processing. The server 202 also includes memory 210 (e.g., random access memory, read-only memory, flash memory); electronic storage unit 215 (e.g. hard disk); communications interface 220 (e.g., network adaptor) for communicating with one or more other systems; and peripheral devices 225 which may include cache, other memory, data storage, and/or electronic display adaptors. The memory 210, storage unit 215, interface 220, and peripheral devices 225 are in communication with the processor 205 through a communications bus (solid lines), such as a motherboard. The storage unit 215 can be a data storage unit for storing data. The server 202 is operatively coupled to a computer network (“network”) 230 with the aid of the communications interface 220. The network 230 can be the Internet, an intranet and/or an extranet, an intranet and/or extranet that is in communication with the Internet, a telecommunication or data network. The network 230 in some cases, with the aid of the server 101, can implement a peer-to-peer network, which may enable devices coupled to the server 202 to behave as a client or a server.

The storage unit 215 can store files, such as output reports, and/or communications with the data about samples, or any aspect of data associated with the present disclosure.

The computer server 202 can communicate with one or more remote computer systems through the network 230. The one or more remote computer systems may be, for example, personal computers, laptops, tablets, telephones, Smart phones, or personal digital assistants.

In some applications the computer system 200 includes a single server 202. In other situations, the system includes multiple servers in communication with one another through an intranet, extranet and/or the internet.

The server 202 can be adapted to store measurement data or a database as provided herein, patient information from the subject, such as, for example, medical history, family history, demographic data and/or other clinical or personal information of potential relevance to a particular application. Such information can be stored on the storage unit 215 or the server 202 and such data can be transmitted through a network.

Methods as described herein can be implemented by way of machine (or computer processor) executable code (or software) stored on an electronic storage location of the server 202, such as, for example, on the memory 210, or electronic storage unit 215. During use, the code can be executed by the processor 205. In some cases, the code can be retrieved from the storage unit 215 and stored on the memory 210 for ready access by the processor 205. In some situations, the electronic storage unit 215 can be precluded, and machine-executable instructions are stored on memory 210. Alternatively, the code can be executed on a second computer system 240.

Aspects of the systems and methods provided herein, such as the server 202, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical, and electromagnetic waves, such as used across physical interfaces between local devices, through wired and optical landline networks and over various air-links. The physical elements that carry such waves, such as wired or wireless likes, optical links, or the like, also may be considered as media bearing the software. As used herein, unless restricted to non-transitory, tangible “storage” media, terms such as computer or machine “readable medium” can refer to any medium that participates in providing instructions to a processor for execution.

The computer systems described herein may comprise computer-executable code for performing any of the algorithms or algorithms-based methods described herein. In some applications the algorithms described herein will make use of a memory unit that is comprised of at least one database.

Data relating to the present disclosure can be transmitted over a network or connections for reception and/or review by a receiver. The receiver can be but is not limited to the subject to whom the report pertains; or to a caregiver thereof, e.g., a health care provider, manager, other health care professional, or other caretaker; a person or entity that performed and/or ordered the analysis. The receiver can also be a local or remote system for storing such reports (e.g. servers or other systems of a “cloud computing” architecture). In one embodiment, a computer-readable medium includes a medium suitable for transmission of a result of an analysis of a biological sample using the methods described herein.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

For purposes of the present invention, “treating” or “treatment” describes the management and care of a subject for the purpose of combating the disease, condition, or disorder. Treating includes the administration of a checkpoint inhibitor therapy when it is determined that the subject would be provided a benefit by the administration of the treatment to prevent the onset of the symptoms or complications, alleviating the symptoms or complications, or eliminating the disease, condition, or disorder.

The term “treating” can be characterized by one or more of the following: (a) the reducing, slowing or inhibiting the growth of cancer, including reducing slowing or inhibiting the growth of cancer cells; (b) preventing the further growth of tumors; (c) reducing or preventing the metastasis of cancer within a patient, and (d) reducing or ameliorating at least one symptom of the cancer. In some embodiments, the optimum effective amounts can be readily determined by one of ordinary skill in the art using routine experimentation.

As used herein, the terms “effective amount” and “therapeutically effective amount” refer to the quantity of active therapeutic agent or agents sufficient to yield a desired therapeutic response without undue adverse side effects such as toxicity, irritation, or allergic response. The specific “effective amount” will, obviously, vary with such factors as the particular condition being treated, the physical condition of the subject, the type of animal being treated, the duration of the treatment, the nature of concurrent therapy (if any), and the specific formulations employed and the structure of the compounds or its derivatives.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, intraaural administration, rectal administration, sublingual administration, buccal administration, and parenteral administration, including injectable such as intravenous administration, intra-arterial administration, intramuscular administration, intradermal administration, intrathecal administration and subcutaneous administration. Administration can be continuous or intermittent. In various aspects, a preparation can be administered therapeutically; that is, administered to treat an existing disease or condition. In a preferred embodiment, the administration is intravenous administration.

The terms “nucleic acid” and “nucleic acid molecule,” as used herein, refer to a compound comprising a nucleobase and an acidic moiety, e.g., a nucleoside, a nucleotide, or a polymer of nucleotides. Nucleic acids generally refer to polymers comprising nucleotides or nucleotide analogs joined together through backbone linkages such as but not limited to phosphodiester bonds. Nucleic acids include deoxyribonucleic acids (DNA) and ribonucleic acids (RNA) such as messenger RNA (mRNA), transfer RNA (tRNA), etc. Typically, polymeric nucleic acids, e.g., nucleic acid molecules comprising three or more nucleotides are linear molecules, in which adjacent nucleotides are linked to each other via a phosphodiester linkage. In some embodiments, “nucleic acid” refers to individual nucleic acid residues (e.g. nucleotides and/or nucleosides). In some embodiments, “nucleic acid” refers to an oligonucleotide chain comprising three or more individual nucleotide residues. As used herein, the terms “oligonucleotide” and “polynucleotide” can be used interchangeably to refer to a polymer of nucleotides (e.g., a string of at least three nucleotides). In some embodiments, “nucleic acid” encompasses RNA as well as single and/or double-stranded DNA. Nucleic acids may be naturally occurring, for example, in the context of a genome, a transcript, an mRNA, tRNA, rRNA, siRNA, snRNA, a plasmid, cosmid, chromosome, chromatid, or other naturally occurring nucleic acid molecule. On the other hand, a nucleic acid molecule may be a non-naturally occurring molecule, e.g., a recombinant DNA or RNA, an artificial chromosome, an engineered genome, or fragment thereof, or a synthetic DNA, RNA, DNA/RNA hybrid, or include non-naturally occurring nucleotides or nucleosides. Furthermore, the terms “nucleic acid,” “DNA,” “RNA,” and/or similar terms include nucleic acid analogs, i.e. analogs having other than a phosphodiester backbone. Nucleic acids can be purified from natural sources, produced using recombinant expression systems and optionally purified, chemically synthesized, etc. Where appropriate, e.g., in the case of chemically synthesized molecules, nucleic acids can comprise nucleoside analogs such as analogs having chemically modified bases or sugars, and backbone modifications. A nucleic acid sequence is presented in the 5′ to 3′ direction unless otherwise indicated. In some embodiments, a nucleic acid is or comprises natural nucleosides (e.g. adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

The present invention has been described in terms of one or more preferred embodiments, and it should be appreciated that many equivalents, alternatives, variations, and modifications, aside from those expressly stated, are possible and within the scope of the invention.

It should be apparent to those skilled in the art that many additional modifications beside those already described are possible without departing from the inventive concepts. In interpreting this disclosure, all terms should be interpreted in the broadest possible manner consistent with the context. Variations of the term “comprising” should be interpreted as referring to elements, components, or steps in a non-exclusive manner, so the referenced elements, components, or steps may be combined with other elements, components, or steps that are not expressly referenced. Embodiments referenced as “comprising” certain elements are also contemplated as “consisting essentially of” and “consisting of” those elements. The term “consisting essentially of” and “consisting of” should be interpreted in line with the MPEP and relevant Federal Circuit interpretation. The transitional phrase “consisting essentially of” limits the scope of a claim to the specified materials or steps “and those that do not materially affect the basic and novel characteristic(s)” of the claimed invention. “Consisting of” is a closed term that excludes any element, step or ingredient not specified in the claim. For example, with regard to sequences “consisting of” refers to the sequence listed in the SEQ ID NO. and does refer to larger sequences that may contain the SEQ ID as a portion thereof.

As used in this specification and the claims, the singular forms “a,” “an,” and “the” include plural forms unless the context clearly dictates otherwise. For example, the term “a substituent” should be interpreted to mean “one or more substituents,” unless the context clearly dictates otherwise.

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean up to plus or minus 10% of the particular term and “substantially” and “significantly” will mean more than plus or minus 10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

The phrase “such as” should be interpreted as “for example, including.” Moreover, the use of any and all exemplary language, including but not limited to “such as”, is intended merely to better illuminate the invention and does not pose a limitation on the scope of the invention unless otherwise claimed.

Furthermore, in those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or ‘B or “A and B.”

All language such as “up to,” “at least,” “greater than,” “less than,” and the like, include the number recited and refer to ranges which can subsequently be broken down into ranges and subranges. A range includes each individual member. Thus, for example, a group having 1-3 members refers to groups having 1, 2, or 3 members. Similarly, a group having 6 members refers to groups having 1, 2, 3, 4, or 6 members, and so forth.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use and aspect of a described embodiment or feature contained in the same, or a definitive decision to use a specific skill regarding a described embodiment or feature contained in the same. In this latter context, the modal verb “may” has the same meaning and connotation as the auxiliary verb “can.”

The invention will be more fully understood upon consideration of the following non-limiting examples.

The present invention has been described in terms of one or more preferred embodiments, and it should be appreciated that many equivalents, alternatives, variations, and modifications, aside from those expressly stated, are possible and within the scope of the invention.

The following Examples are offered for illustrative purposes only, and are not intended to limit the scope of the present invention in any way. Indeed, various modifications of the invention in addition to those shown and described herein will become apparent to those skilled in the art from the foregoing description and the following examples and fall within the scope of the appended claims.

### EXAMPLES

### Example 1: A Gene Expression Signature of TREM2^{hi} Macrophages and γδ T Cells Predicts Immunotherapy Response

### Introduction

While immune checkpoint therapies (ICT) have improved outcomes for some cancer patients, most patients do not respond to ICT. Previous whole-exome sequencing (WES) and transcriptome sequencing of tumors identified multiple factors that are associated with favorable ICT outcome, including expression of PD-L1¹, high tumor mutational burden², and the presence of tumor-infiltrating CD8⁺ T cells³. Markers indicative of unfavorable response include defects in IFNγ pathways or antigen presentation^{4,5}. While these studies represented a first step in identifying biomarkers, studies using single-cell RNA sequencing (scRNA-seq) have the potential to greatly improve the identification of factors underlying ICT outcomes. For example, one scRNA-seq study of 48 tumor biopsies of responding and non-responding tumors after ICT treatment has the potential to be insightful given the number of patients and high quality data⁶.

To determine if some types of immune cells and their subclusters are associated with ICT outcomes, we analyze the scRNA-seq datasets from multiple outstanding studies^{6_8} and identify the immune cell subpopulations that could play an important role in determining ICT responsiveness. The analysis of several additional bulk RNA-seq datasets of melanoma^{9_12} identifies and validates an ICT outcome signature—ImmuneCells.Sig—enriched with the genes characteristic of the immune cell subsets detected in the scRNA-seq studies. It predicts the ICT outcomes of melanoma patients more accurately than the previously reported ICT response signatures.

Specifically, we find that a subset of macrophages (cluster 12) and a subset of gammadelta (γδ) T cells (cluster 21) are highly enriched in the ICT non-responding tumors. On the other hand, the percentage of a subset of B cells (cluster 22) is significantly smaller in the ICT non-responders compared to the responders. The validated ImmuneCells.Sig ICT outcome signature is enriched with the genes characteristic of the above three immune cell subsets. It can predict the ICT outcomes of melanoma patients more accurately than the previous outstanding signatures, thereby supporting the role of these specific types of immune cells in affecting the ICT outcomes. These findings substantially extend our understanding of the factors associated with ICT responsiveness. Our results may warrant further investigation in the cancer immunotherapy setting.

### Results

Association of Immune Cell Populations with ICT Outcome

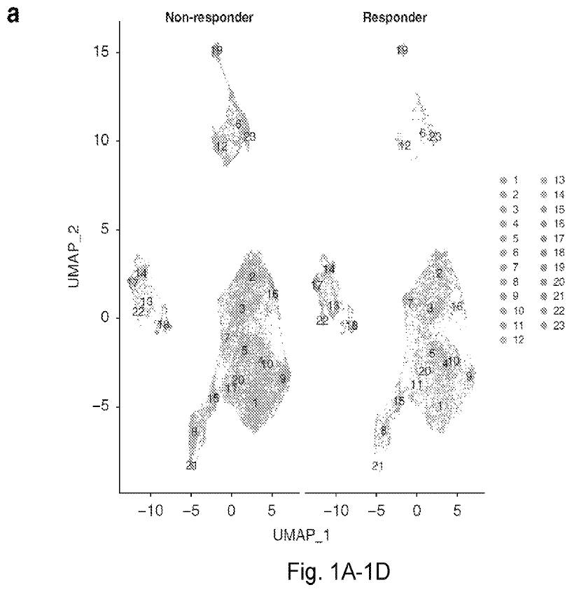

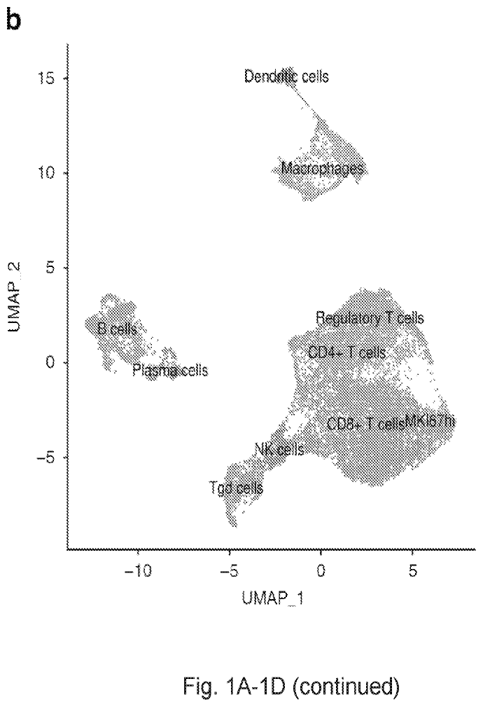

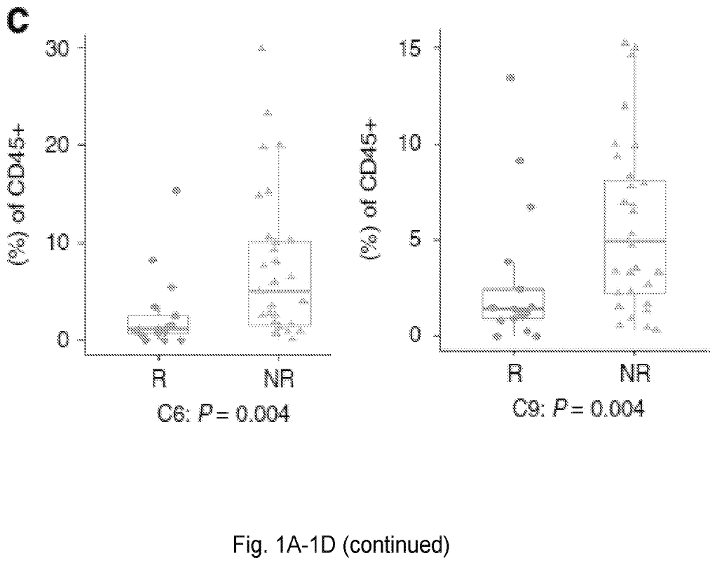

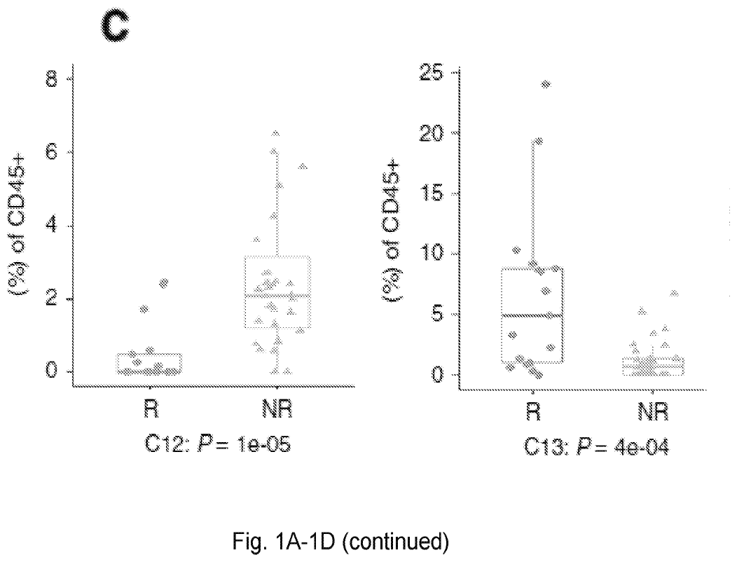

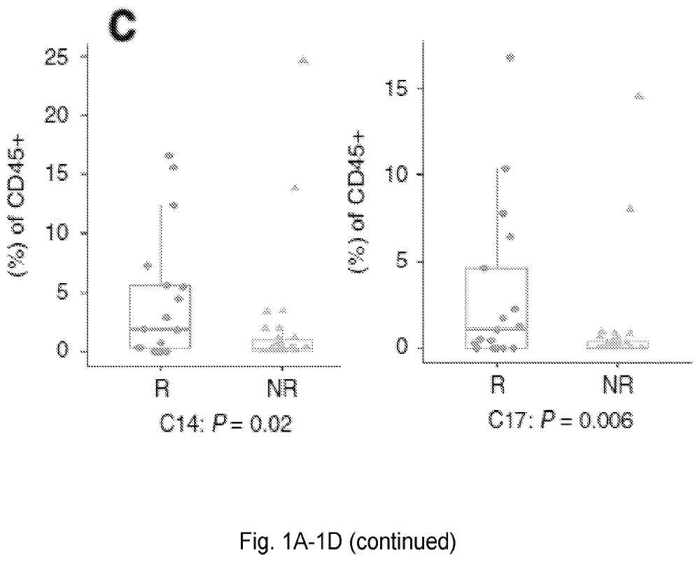

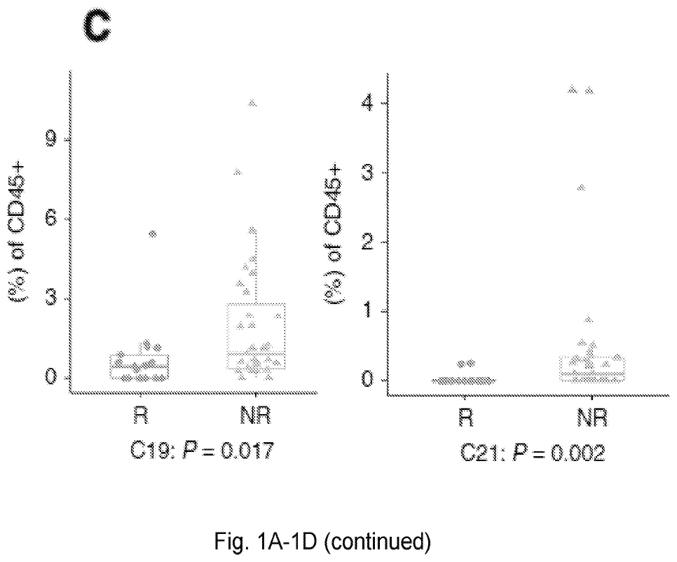

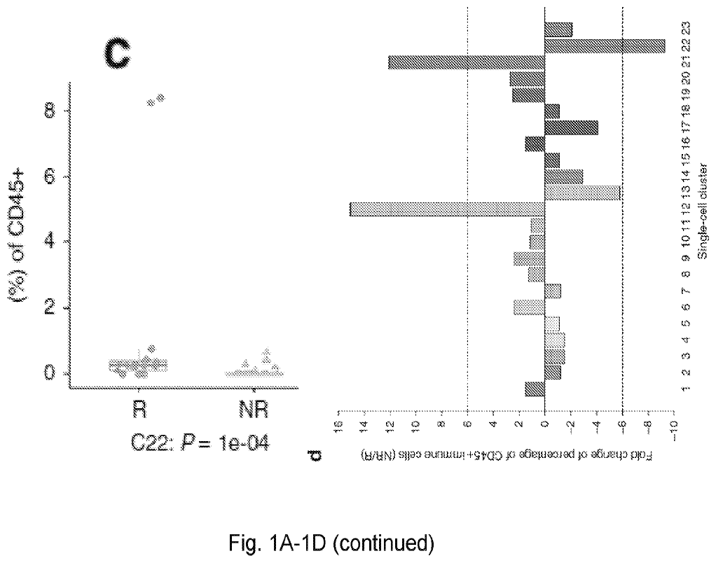

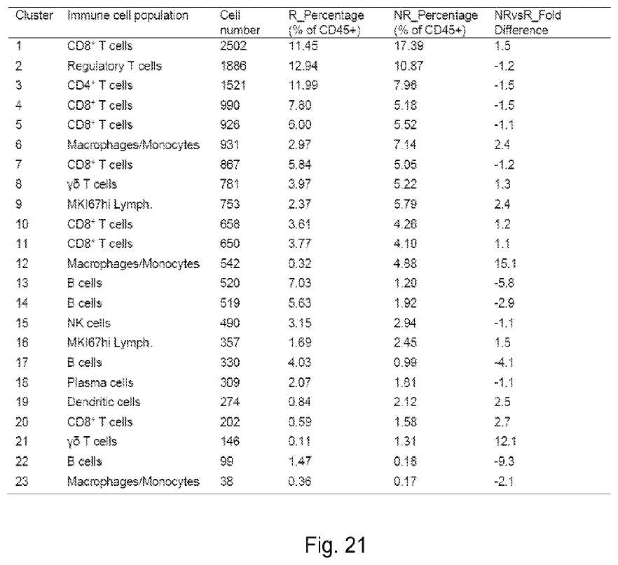

We utilized the Seurat package^{13,14} to perform fine clustering of the original 16,291 single cells based on raw data from a previous melanoma study⁶. The melanoma patient response categories were defined by RECIST (Response evaluation criteria in solid tumors) as: complete response (CR) and partial response (PR) for responders, or stable disease (SD) and progressive disease (PD) for non-responders¹⁵. Progression-free survival was also considered in distinguishing the responders from non-responders. To relate molecular and cellular variables with responses of individual lesions to therapy, the previous study classified each of the 48 tumor samples based on radiologic assessments into progression/non-responder (NR; n=31, including SD/PD samples) or regression/responder (R; n=17, including CR/PR samples)⁶. The gene expression data of single cells from tumors with different ICT outcomes, i.e., regression/responder (Responder—‘R’; n.patients=17; n.cells=5564) and progression/non-responder (Non-Responder—‘NR’; n.patients=31; n.cells=10,727), were aligned and projected in two-dimensional space through uniform manifold approximation and projection (UMAP)¹⁶ to allow the identification of ICT-outcome-associated immune cell populations. This analysis generated 23 cell clusters across all samples (FIG. 1a). The percentages of immune cells from each cluster from responding and non-responding melanoma groups were calculated (FIG. 21).

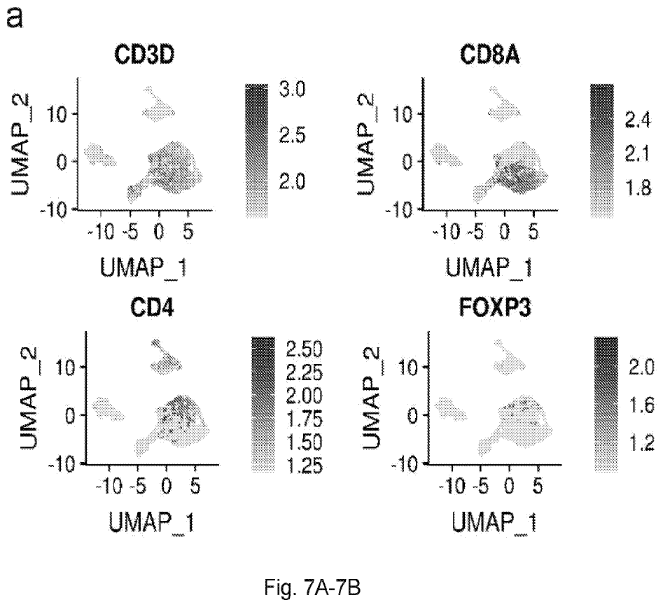

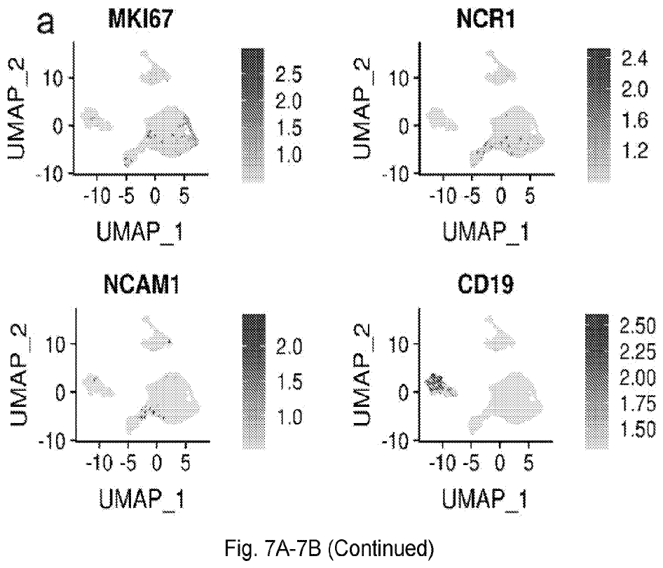

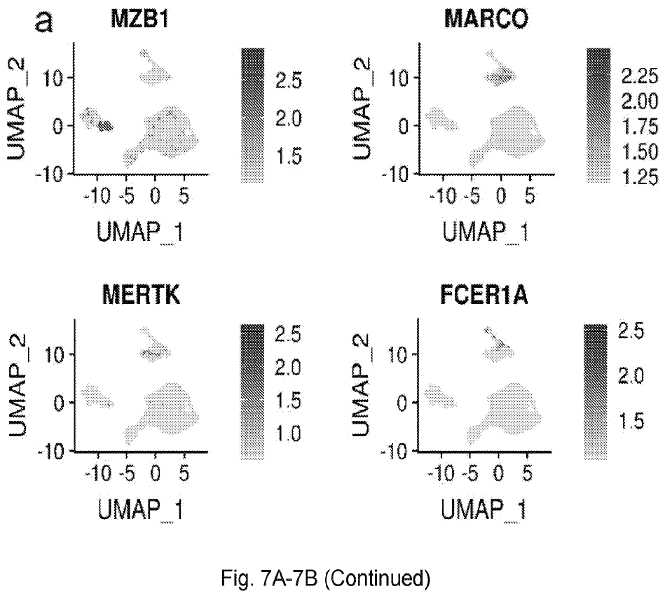

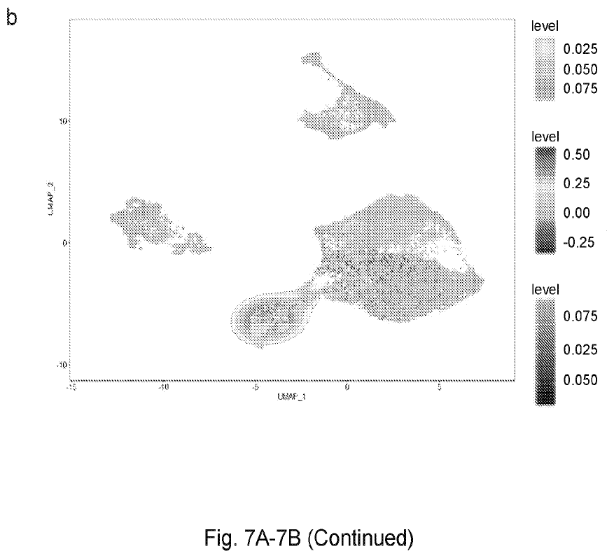

We utilized gene expression patterns of canonical markers to classify the 23 clusters into 10 major immune cell populations (FIG. 1b and FIG. 7a): CD8⁺ T cells (CD3⁺CD8A⁺CD4⁻, clusters 1, 4, 5, 7, 10, 11, 20); CD4⁺ T cells (CD3⁺CD8A⁻CD4⁺, cluster 3); Regulatory T cells (Tregs) (FOXP3⁺, cluster 2); MKI67hi Lymph. (MKI67⁺, clusters 9,16); B cells (CD19⁺, clusters 13, 14, 17, 22); Plasma cells (MZB1⁺, cluster 18); NK cells (NCR1⁺NCAM1⁺, cluster 15); γδ T cells (i.e., Tgd cells, CD3⁺CD8A⁻CD4⁻, clusters 8,21); Macrophages (MARCO⁺MERTK⁺, clusters 6, 12, 23); and Dendritic cells (FCER1A⁺, cluster 19). The identification of γδ T cells is further justified as follows. The NK cells in cluster 15 expressed the NK cell markers NCR1 and NCAM1, which were not expressed in γδ T cells in clusters 8 and 21 (FIG. 7a). Also, the NK cells (cluster 15) do not express CD3 markers, whereas CD3 markers were expressed in the adjacent clusters (8 and 21) that were characterized as γδ T cells based on the combination CD3⁺CD4⁻CD8⁻. In addition, we validated our defined γδ T lymphocytes by the expression of the published gene expression signatures of γδ T cells¹⁷, which requires scoring the following two gene sets: the positive gene set (CD3D, CD3E, TRDC, TRGC1, and TRGC2), and the negative gene set (CD8A and CD8B) for each single cell. Specifically, following this published approach, to identify γδ T lymphocytes exhaustively and without NK and T-cell CD8 false-positives, we utilized the established γδ signature that combines the above two gene sets that were scored for each single cell and visualized in the UMAP by Single-Cell Signature Explorer¹⁸. As shown in FIG. 7b, the γδ signature scores were highest for clusters 8 and 21 but much lower in the other clusters. These data further support our assignment of γδ T lymphocytes to clusters 8 and 21.

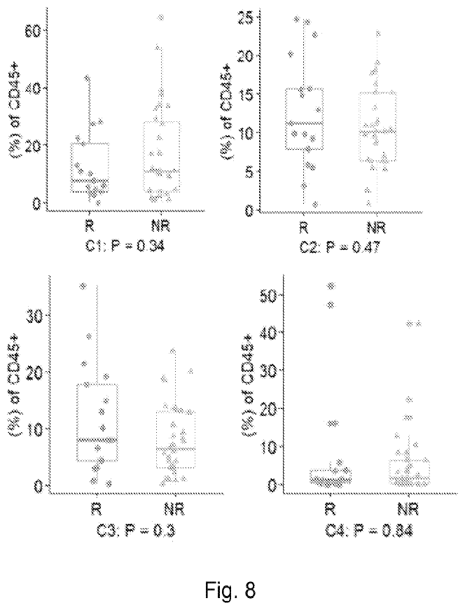

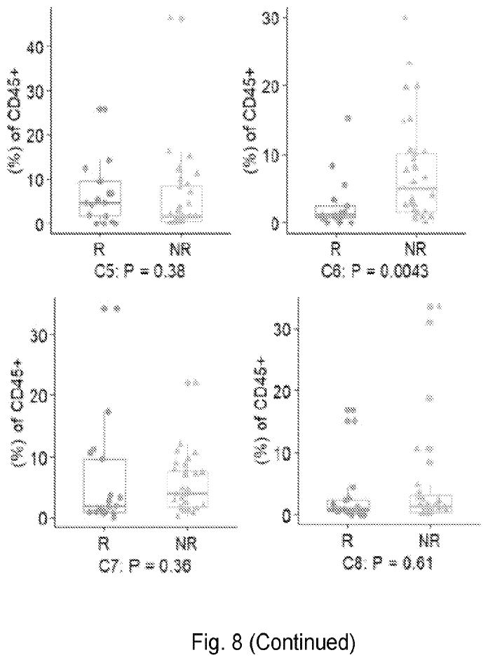

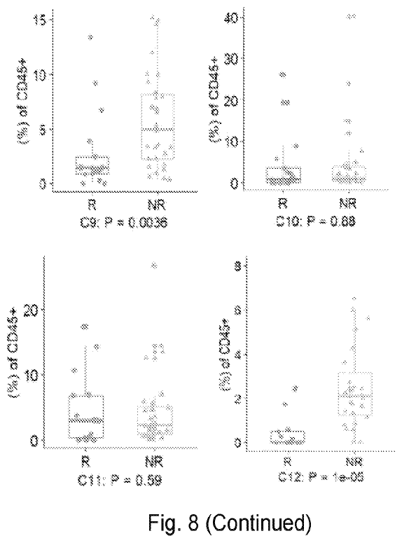

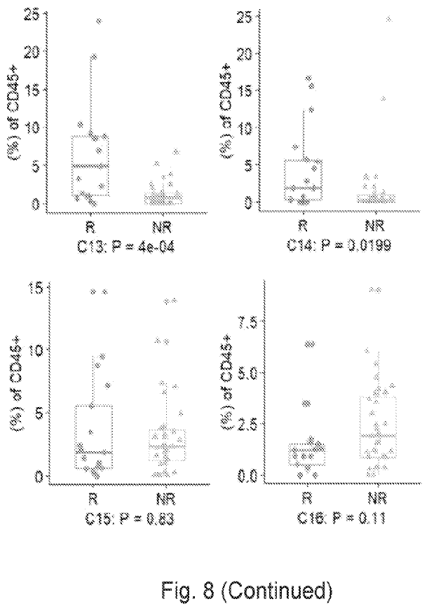

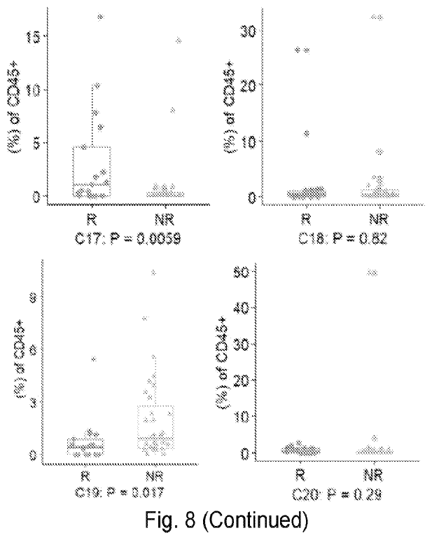

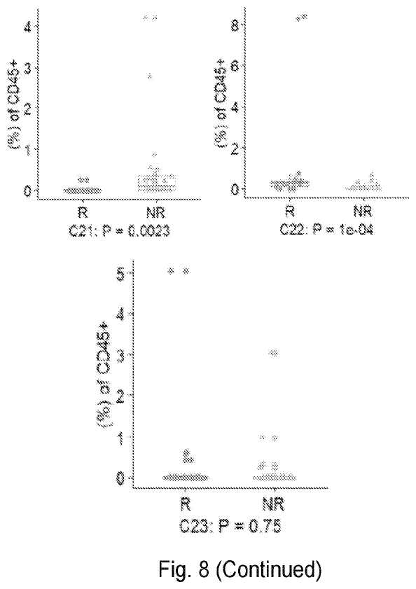

We tested the 23 immune cell clusters for their percentage differences between the non-responders and responders at the patient level (FIG. 1c and FIG. 8). The results were compared to the results of the integrative analysis to calculate the overall fold changes between the non-responder and responder groups. Some of these immune cell clusters differed quantitatively between ICT responders (R) and non-responders (NR), including the Clusters 6, 9, 12, 13, 14, 17, 19, 21, 22 (FIG. 1c), which was supported by the integrative analysis combining cells from all patients (FIG. 1d). Furthermore, using more than 6-fold differences as a biologically significant threshold¹⁹, we identified three clusters (12, 21, and 22) that exceeded this criterion. Cluster 12 (a macrophage cluster) and cluster 21 (a γδ T-cell cluster) cells were 15.1-fold and 12.1-fold higher, respectively, in ICT non-responders versus responders (FIG. 1d and FIG. 21). In contrast, the percentage of cluster 22 cells (a B-cell cluster) was 9.3-fold lower in the non-responders. Two other B-cell clusters (cluster 13 and 17) were 5.8- and 4.1-fold lower, respectively, in the non-responders. The remaining 18 clusters exhibited only minor (1.1- to 2.9-fold) differences between responders non-responders (FIG. 1d and FIG. 21). Our approach is similar to the approach used in the previous scRNA-seq study of the effects of the immunotherapy on changing the percentages of different immune cell subpopulations²⁰. They compared the percentage of cells in individual clusters for different conditions of control, anti-PD-1, anti-CTLA-4, and anti-PD-1/anti-CTLA-4. In this way, they identified a number of immune cell subclusters that could be associated with the variation of the efficacy of the cancer immunotherapy.

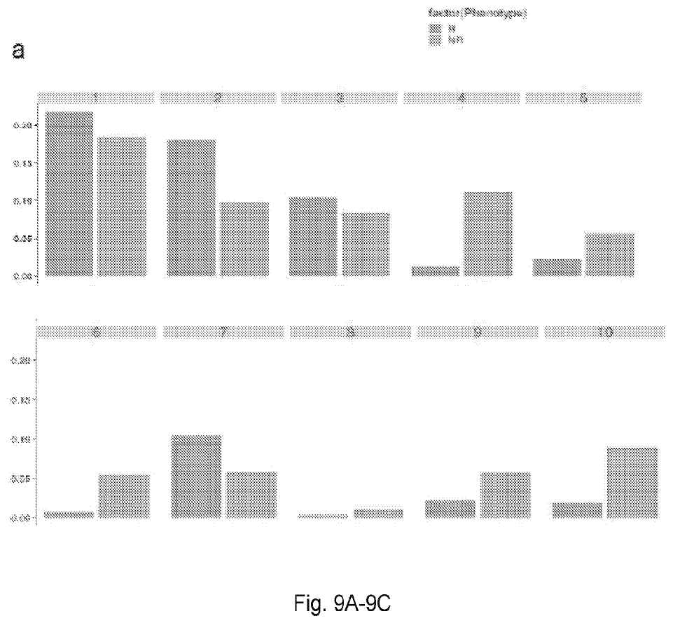

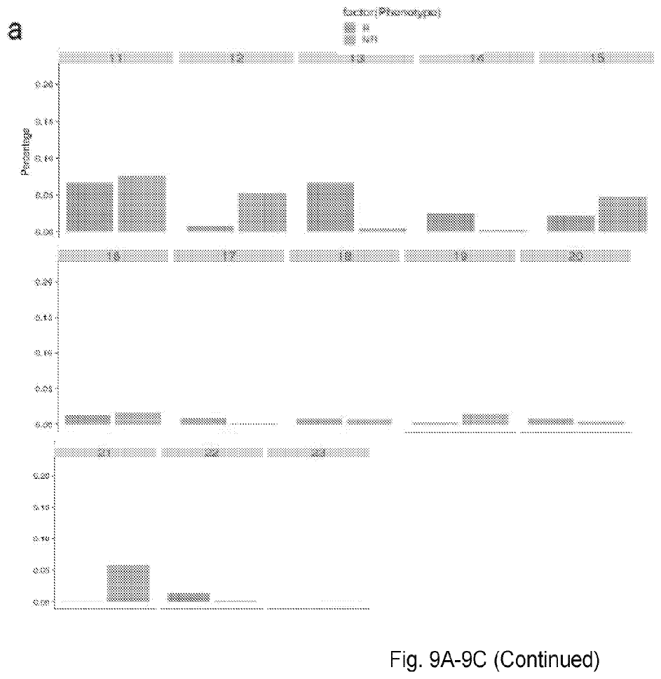

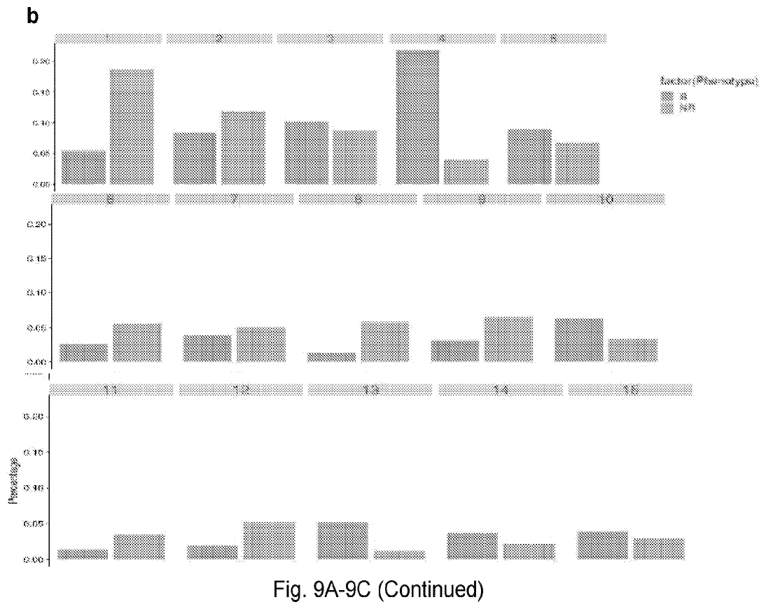

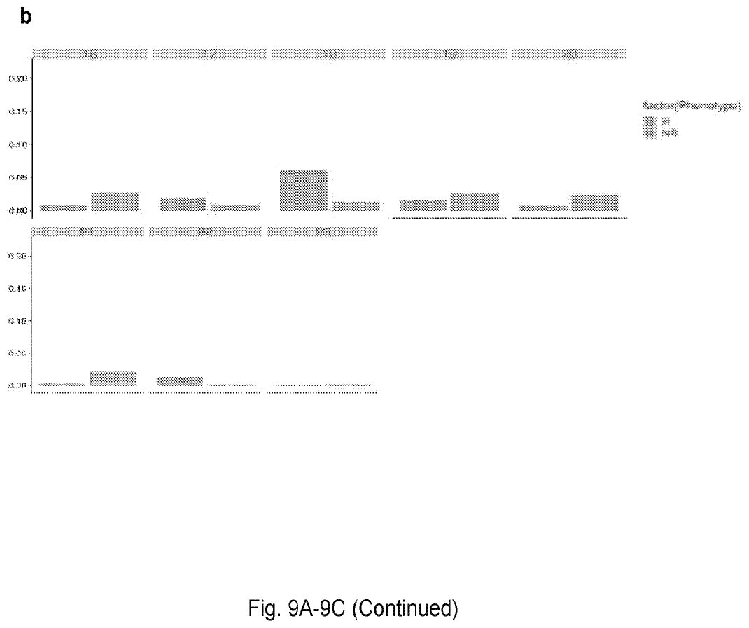

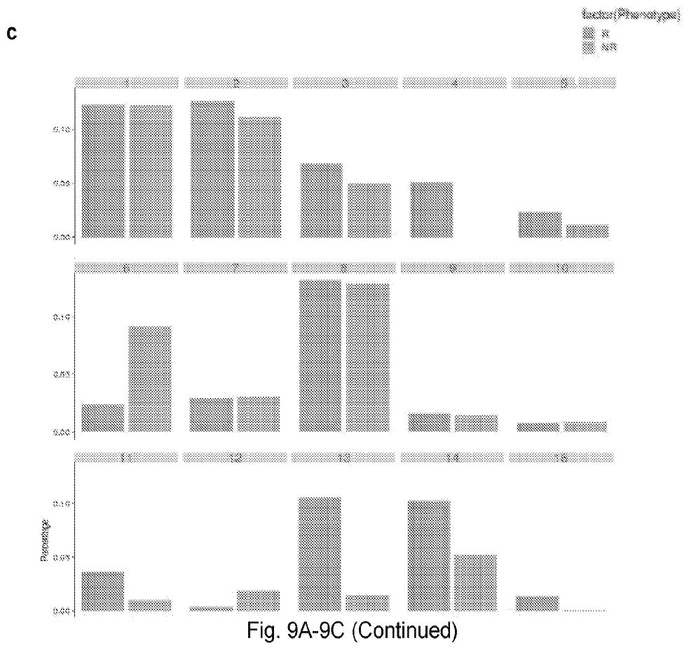

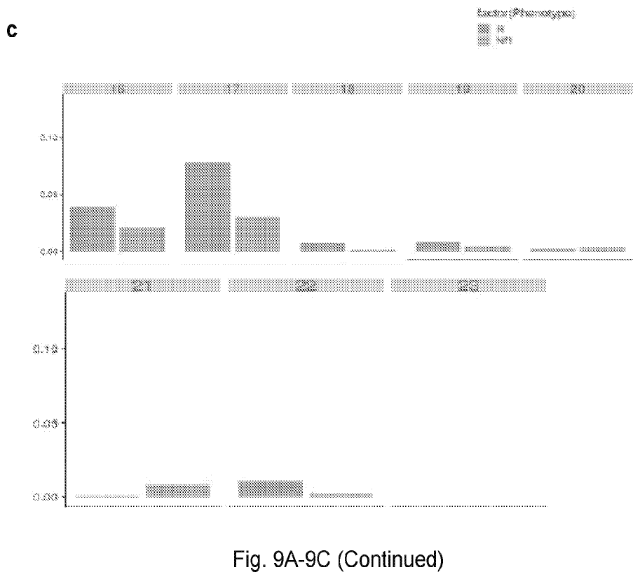

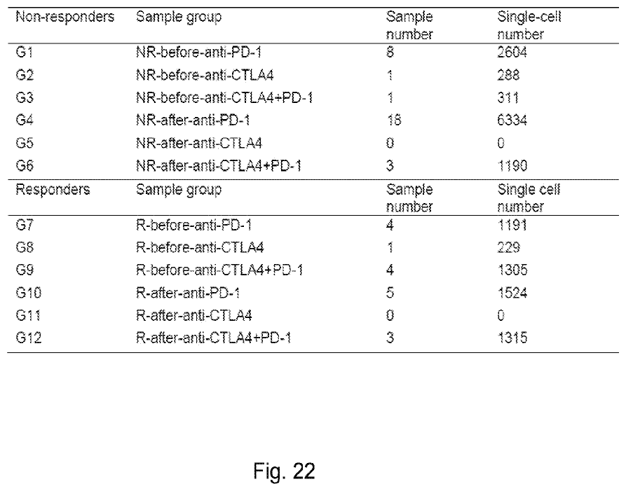

To account for clinical differences, we divided the melanoma samples into subgroups according to three factors: (1) ICT outcomes, (2) sample collection time (before or after ICT), and (3) treatment schemes (FIG. 22). There were only six groups with sufficient numbers of samples and cells to compare between non-responders and responders, i.e., G1 vs G7 (‘NR-before-anti-PD-1’ vs ‘R-before-anti-PD-1’), G4 vs G10 (‘NR-after-anti-PD-1’ vs ‘R-after-anti-PD-1’), and G6 vs G12 (‘NR-after-anti-CTLA4+PD-1’ vs ‘R-after-anti-CTLA4+PD-1’). Stratified analyses showed similar results of cell cluster percentage changes as those in the integrative analysis (FIG. 1d and FIG. 9).

TREM2^{hi} Macrophages May Contribute to ICT Resistance

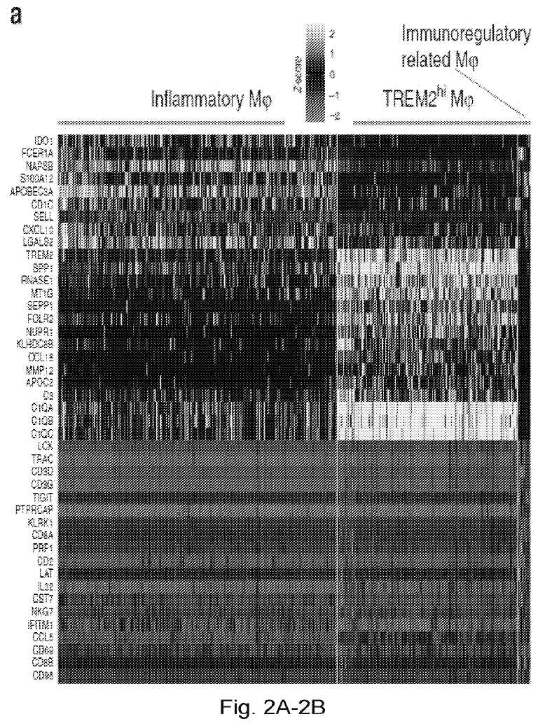

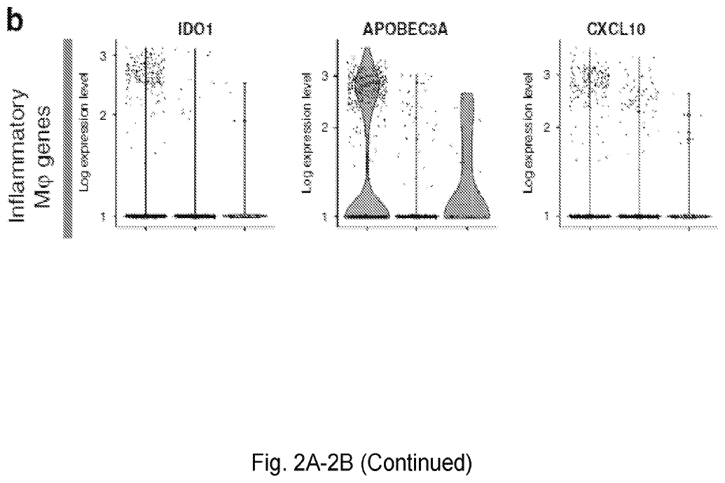

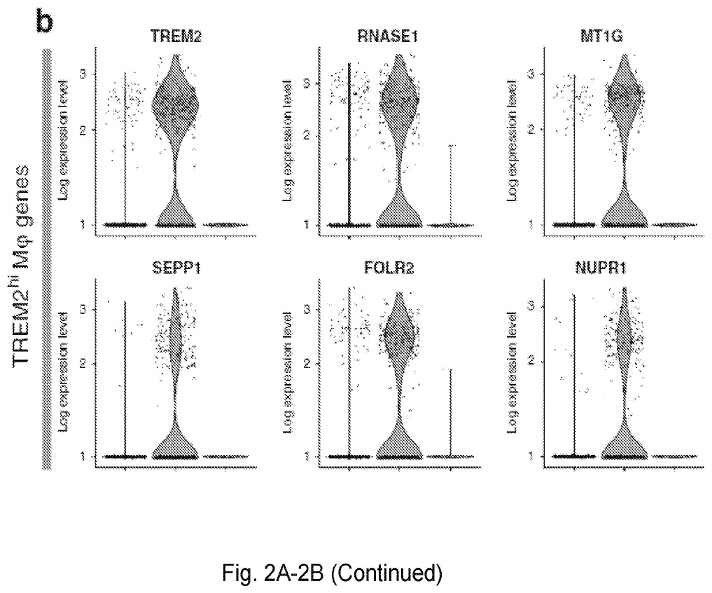

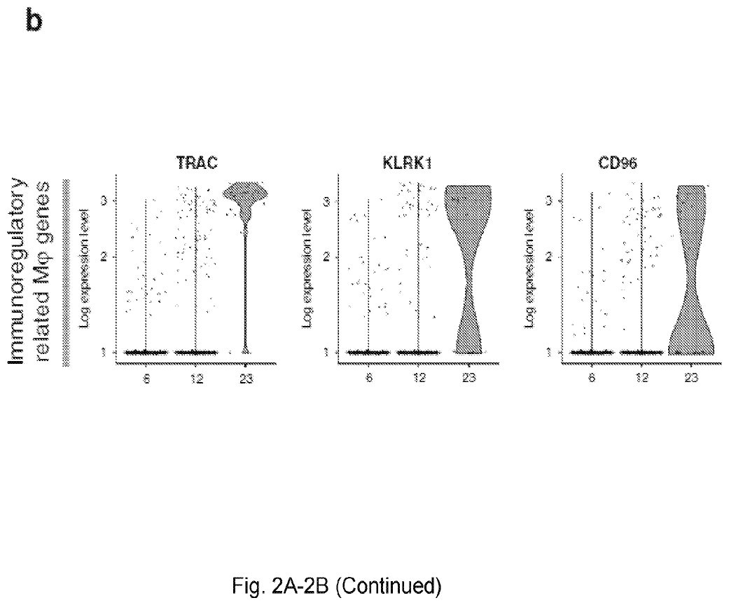

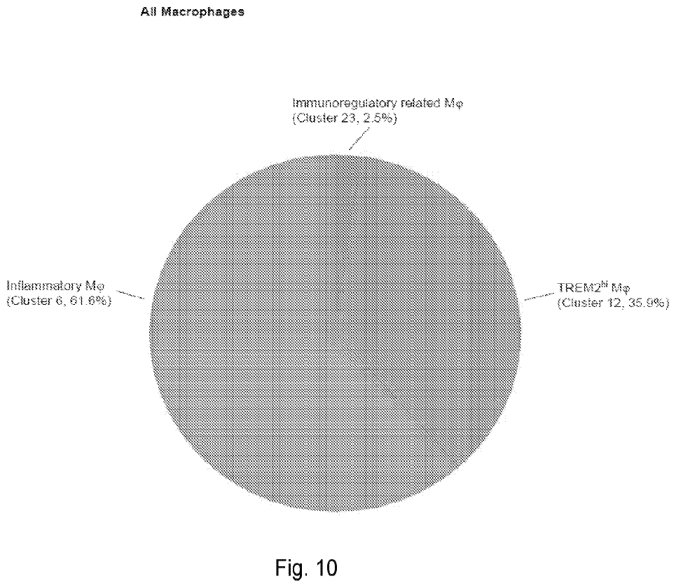

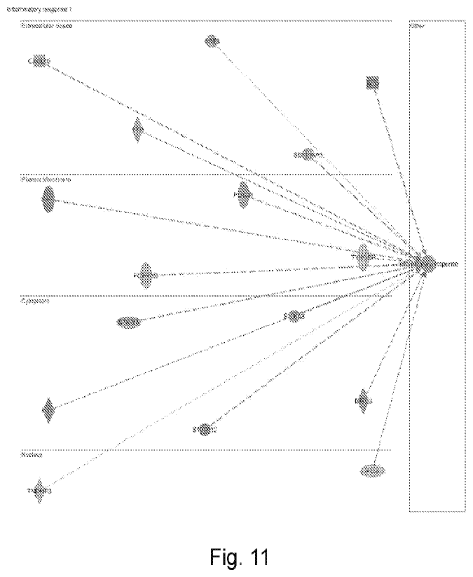

Of the macrophage populations in clusters 6, 12, and 23 (FIG. 1a, b), differences between the R and NR groups were not significant for cluster 6 (2.4-fold higher in NR) and cluster 23 (2.1-fold lower in NR). However, cluster 12 macrophages were 15.1-fold higher in NR (4.88%) versus R (0.32%). This enrichment of cluster 12 in non-responders suggests that it may be associated with ICT resistance. Single-cell differential expression analyses were performed to assess the most characteristic gene expression differences in clusters 6, 12, and 23 (FIG. 2 and FIGS. 8, 9, and 10). Cluster 12 (35.9% of all macrophages, FIG. 10) overexpressed TREM2 (FIG. 2) so was named as TREM2^{hi} Mφ (Mφ=macrophages). The TREM2^{hi} Mφ that were enriched in non-responders displayed a unique signature with overexpression of SPP1, RNASE1, MT1G, SEPP1, FOLR2, NUPR1, KLHDC8B, CCL18, MMP12, and APOC2 along with key complement system genes (C3, C1QA, C1QB, and C1QC) (FIG. 2). Cluster 6 cells (61.6% of all Mφ, FIG. 10), overexpressed the immunosuppressive protein indoleamine 2,3-dioxygenase 1 (IDO1) (FIG. 2), as well as several inflammatory markers (FCER1A, S100A12, APOBEC3A, SELL, and CXCL10). Ingenuity Pathway Analysis (IPA) confirmed that inflammatory markers were significantly overexpressed in cluster 6 versus other macrophages (adjusted P=3.93E−10, activation Z score=2.01, FIG. 11; P values throughout this paper are adjusted by using Bonferroni correction unless otherwise declared). Therefore, we named cluster 6 as Inflammatory Mφ. Cluster 23 cells (2.5% of all Mφ, FIG. 10) were 2.1-fold higher in responders and expressed several genes involved in immune regulation, i.e., LCK, TIGIT, PTPRCAP, KLRK1, LAT, IL32, IFITM1, and CCL5 (FIG. 2a)²¹. Cluster 23 was thus named as Immunoregulatory related Mφ.

Significantly Enriched Pathways in TREM2^{hi} Macrophages

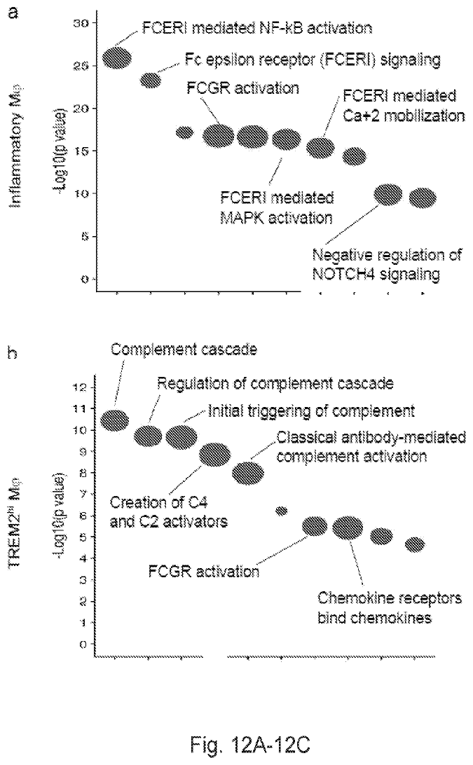

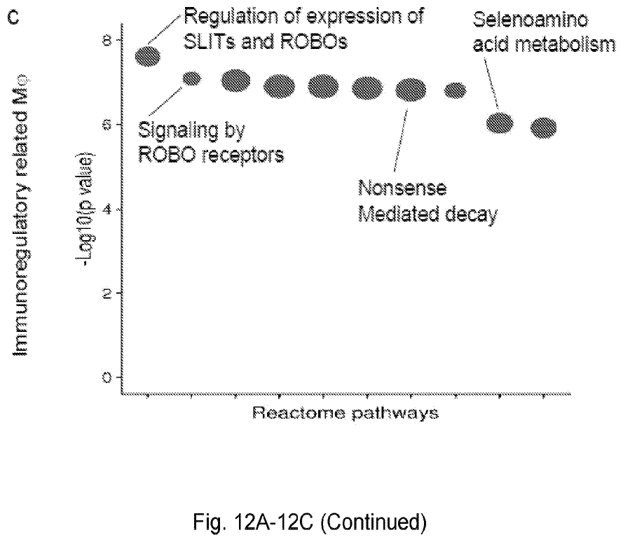

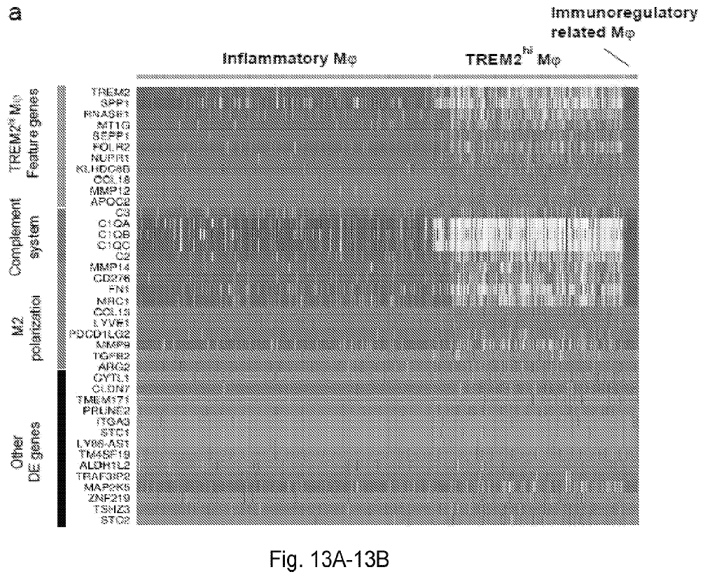

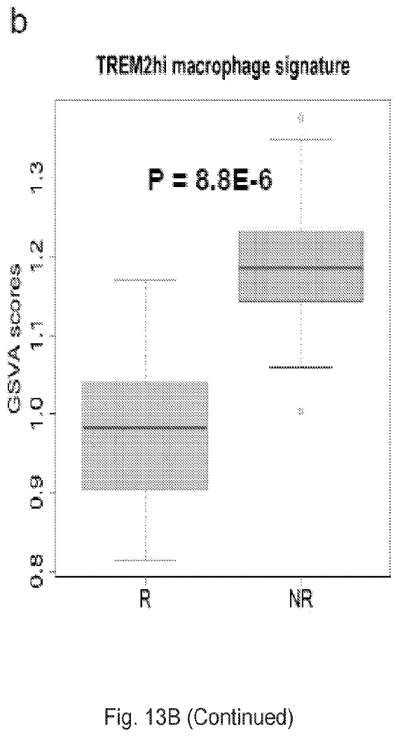

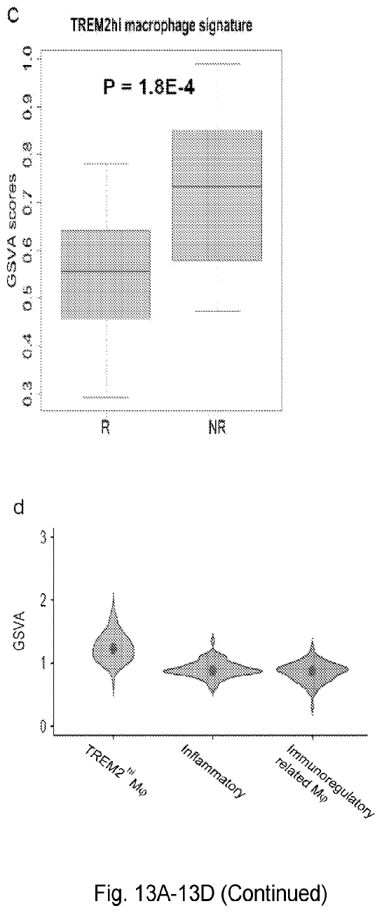

To identify if functional heterogeneity of these macrophage subsets could be associated with ICT outcomes, we performed ‘Reactome pathways’ analysis for macrophages based on cluster-specific genes detected by Seurat (FIGS. 11, 12, and 13). Each macrophage subset was significantly enriched for specific molecular pathways. Inflammatory Mφ (cluster 6) were enriched for FCERI signaling and several FCERI-mediated pathways (NF-kappaB activation, Ca²⁺ mobilization and MAPK activation; FIG. 12). The Immunoregulatory related Mφ (cluster 23) were most significantly enriched for pathways involving Regulation of expression of SLITs and ROBOs and Signaling by ROBO receptors (FIG. 12). TREM2^{hi} Mφ (cluster 12), which showed the greatest percentage elevation in ICT non-responders, was enriched for multiple pathways underlying complement activation (complement cascade and its regulation, initial triggering of complement, creation of C4 and C2 activators, and classical antibody-mediated complement activation; FIG. 12). These findings were consistent with overexpression of complement system genes in TREM2^{hi} Mφ, including complement C1q chains (C1QA, C1QB, and C1QC), complement C2 and C3 (FIG. 13a). These genes were either not expressed, or at very low levels in macrophage clusters 6 and 23. TREM2^{hi} macrophages also overexpressed M2 polarization genes (MMP14, CD276, FN1, MRC1, CCL13, CCL18, LYVE1, PDCD1LG2 (PD-L2), MMP9, TGFB2, and ARG2; FIG. 13a). TREM2^{hi} macrophages may therefore be functionally proximal to M2 polarization macrophages and could block the anti-tumor activities of ICT and contribute to ICT resistance.

Validation of the TREM2^{hi} Macrophage Signature

Since TREM2^{hi} macrophages correlated with ICT resistance, we determined if tumors enriched in TREM2^{hi} macrophages were associated with poor ICT outcomes. Based on the overexpressed genes of this macrophage subset, we developed a 40-gene set to characterize TREM2^{hi} macrophages, which included the genes highly correlated with TREM2 expression (those for the complement system or M2 polarization), and other overexpressed genes (FIG. 13a). In order to test if this TREM2^{hi} macrophage signature was correlated with ICT resistance, we analyzed two publicly available gene expression datasets of tumor samples from melanoma patients treated with immunotherapy^{9,10}. The GSVA scores of the TREM2^{hi} macrophage geneset were significantly higher in the ICT non-responders than the responders (FIG. 13b, c), suggesting that melanomas in non-responders were enriched for TREM2^{hi} macrophages. The analyses of the GSVA scores of this 40-gene set verified the specificity of this gene set to characterize the TREM2^{hi} macrophages among all groups of macrophages (FIG. 13d).

Association of γδ T- and B-Cell Subsets with ICT Outcome

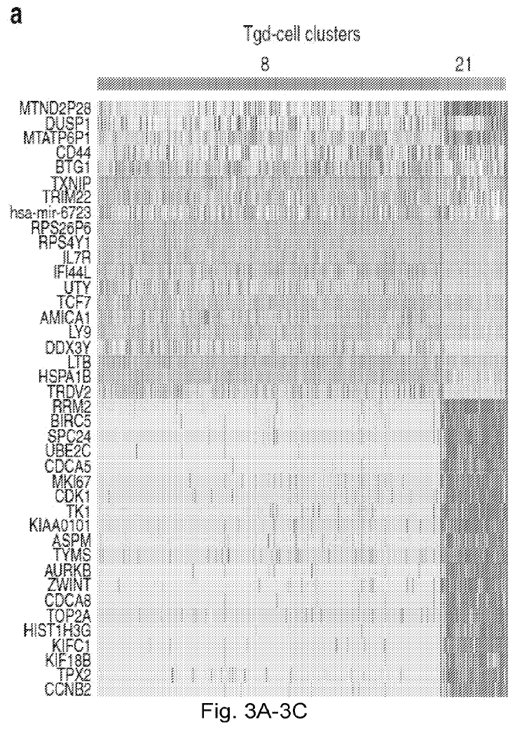

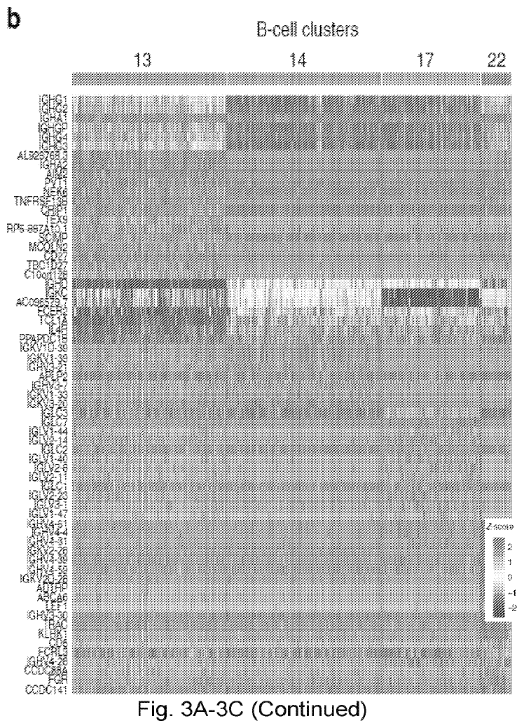

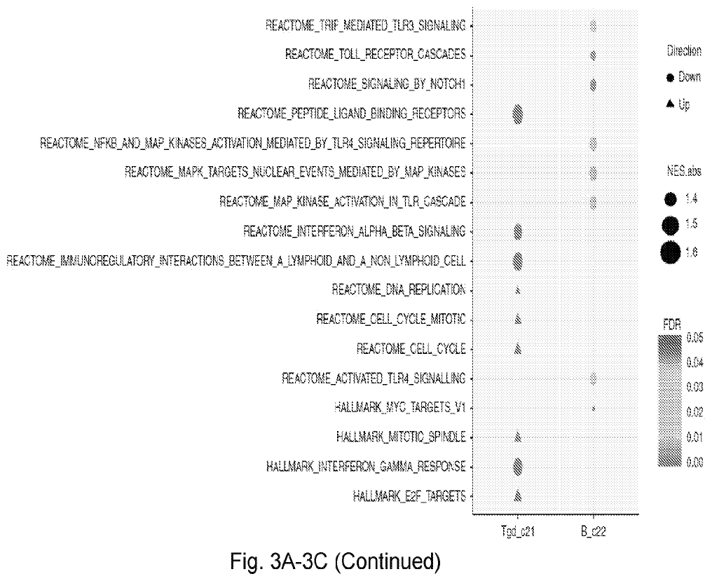

We also identified two clusters of γδ T cells (927 cells total; clusters 8 and 21, FIG. 1, and FIG. 21). The more common type of γδ T cells (cluster 8, n=781) was not significantly different between non-responders and responders. However, a rare type of γδ T cells (cluster 21, n=146) was 12.1-fold higher in the NR group (1.31% versus 0.11% in R; FIG. 1 and FIG. 21). This fold-difference is the second largest of all 23 clusters. These findings suggest that the cluster 21 γδ T cells (named as Tgd_c21) may contribute to ICT resistance. Single-cell differential expression analyses compared Tgd_c21 to Tgd_c8 cells (FIG. 13), with the top 20 marker genes shown in FIG. 3a. The top Tgd_c21 marker genes included RRM2, BIRC5, SPC24, UBE2C, and CDCA5. GSEA pathway analyses²² revealed multiple pathway changes that could be correlated with the contribution of Tgd_c21 cells to ICT resistance, including significant reductions in ligand-receptor binding capacity, IFNα and IFNβ signaling, IFN-γ response, and immunoregulatory interactions (FIG. 3c). Oncogenic (HALLMARK_E2F_TARGETS) and cell cycle pathways were also activated in Tgd_c21 (FIG. 3c). Thus, Tgd_c21 cells may represent a previously unidentified class of γδ T cells that may impair anti-tumor immune functions.

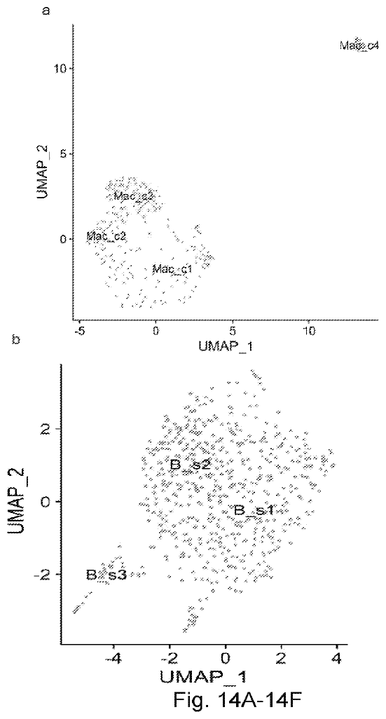

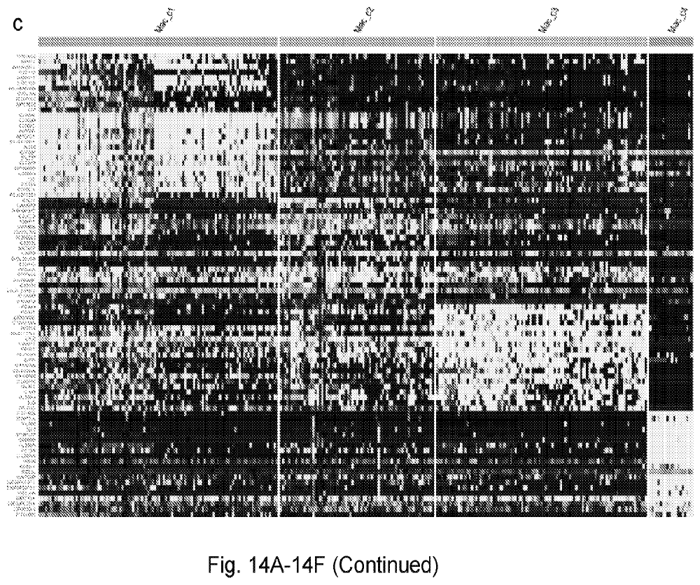

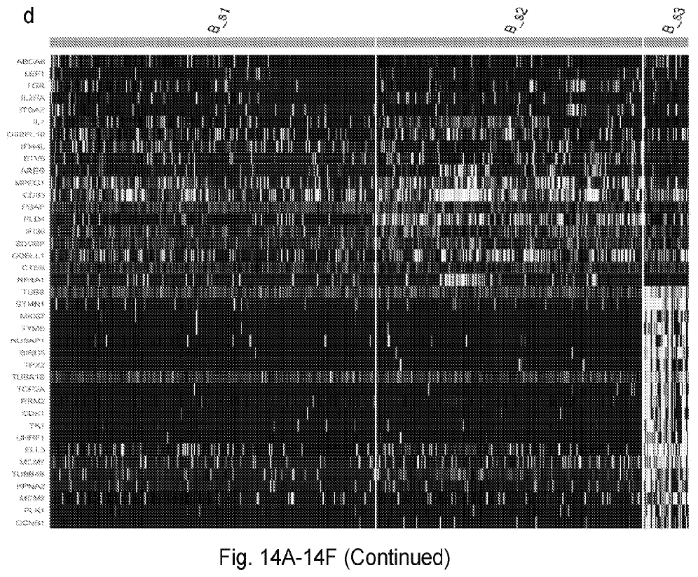

We also identified a correlation between the presence of B cells and ICT response. All four B-cell clusters (13, 14, 17, and 22) were less abundant in the ICT non-responders, which suggests that tumor-associated B cells, in general, are associated with favorable ICT response. Most notably, the percentage of cluster 22 B cells (named as B_c22) was 9.3-fold lower in NR versus R (FIG. 1 and FIG. 21); this is the largest deficit in NR tumors across all 23 clusters. We performed differential expression and pathway enrichment analyses comparing B_c22 to other B-cell clusters (FIG. 14). The top 20 marker genes for each B-cell cluster were determined (FIG. 3b). GSEA pathway analysis showed that B_c22 cells had significantly reduced oncogenic signaling, including Toll receptor signaling/cascades, NOTCH1, MAPK, and MYC signaling pathways (FIG. 3c). The significant enrichment of B_c22 cells in ICT responders may therefore contribute to the attenuation of oncogenic signaling in the tumor microenvironment (TME) to enhance the anti-tumor effect in response to ICT.

Validation in the Other scRNA-Seq Datasets of ICT Patients

To validate the results we found based on the initial scRNA-seq data, we downloaded and re-analyzed another scRNA-seq dataset of melanoma with corresponding immunotherapy efficacy data⁷. This dataset did not have γδ T-cell data available. Interestingly, the deeper clustering of the macrophages and B cells sequenced by this study showed the existence of similar macrophage and B-cell subpopulations that resemble our identified TREM2^{hi} macrophages and B_c22 B cells (FIG. 14a, b). Specifically, the ‘Mac_c1’ macrophage subcluster overexpressed the TREM2^{hi} macrophage marker genes such (TREM2, SPP1, RNASE1, MT1G, SEPP1, FOLR2, KLHDC8B, CCL18, MMP12, APOC2, C3, C 1QA, C1QB, and C1QC; FIG. 14c); the ‘B_s1’ B-cell subcluster overexpressed the B_c22 B cell marker genes (ABCA6, LEF1, FGR, IL2RA, ITGAX, and IL7) (FIG. 14d). More importantly, we validated the behavior of these two immune cell subpopulations in the context of the response to immunotherapy. We scored each cell based on its overall expression (OE) of the corresponding signature following the previous approach⁷, i.e., scoring each Mac_c1 macrophage for its TREM2^{hi} macrophage signature and each B_s1 B cell for its B_c22 B-cell signature, and compared these between the non-responder and control groups. In this dataset, the Mac_c1 macrophage subset had significantly higher overall expression of the TREM2^{hi} macrophage signature in the immunotherapy non-responders than in the control samples (FIG. 14e). The B_s1 B-cell subset had significantly lower overall expression of the B_c22 B-cell signature in the immunotherapy non-responders than in the control samples (FIG. 14f). These results supported the findings in our initial scRNA-seq dataset of the changes in TREM2^{hi} macrophages and B_c22 B cells in response to immunotherapy.

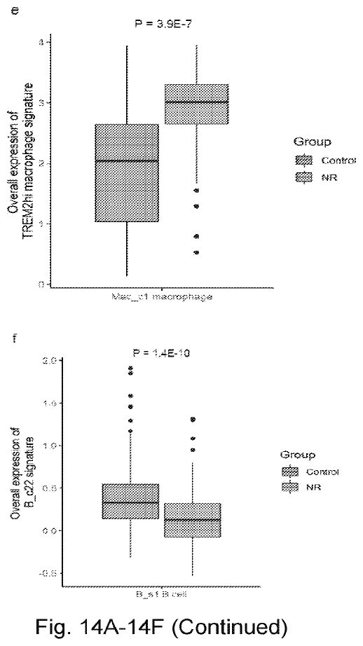

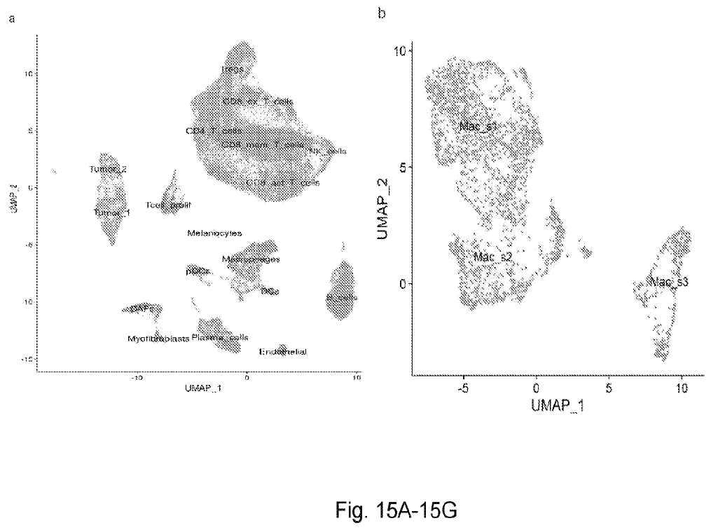

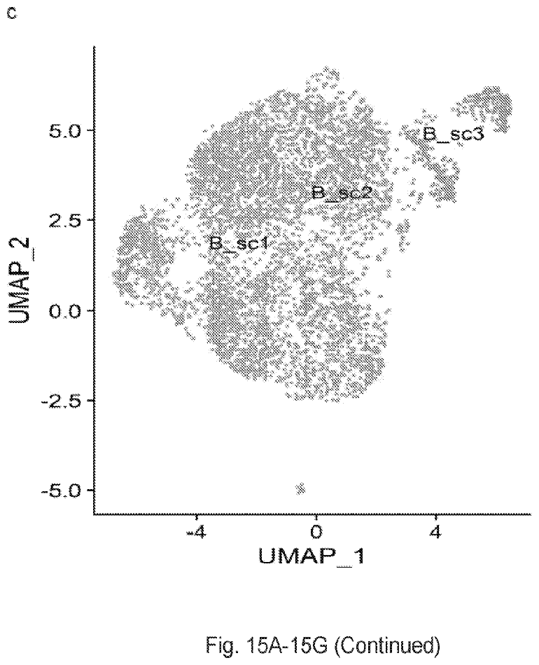

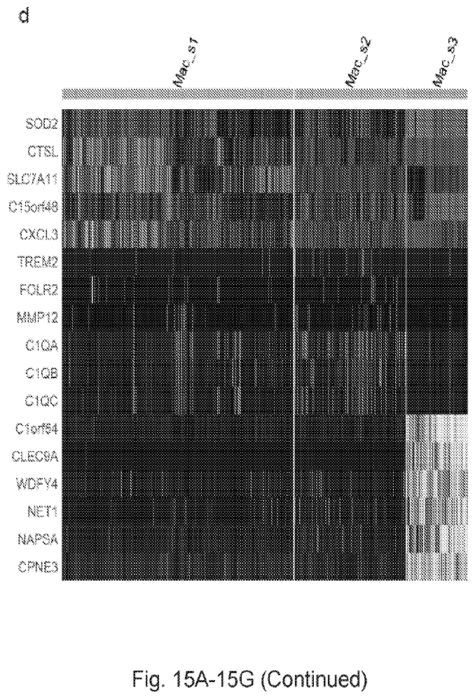

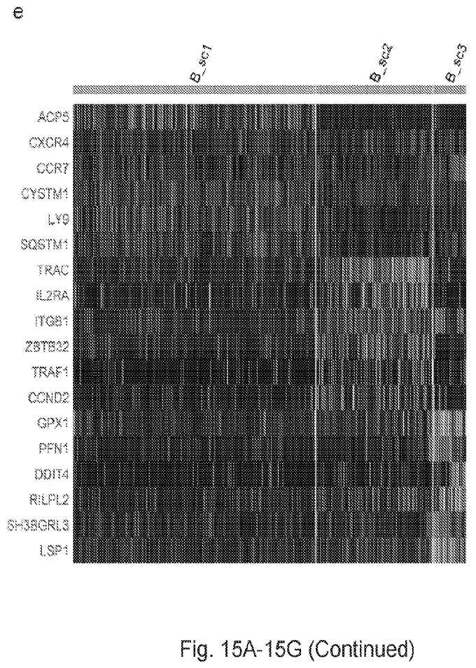

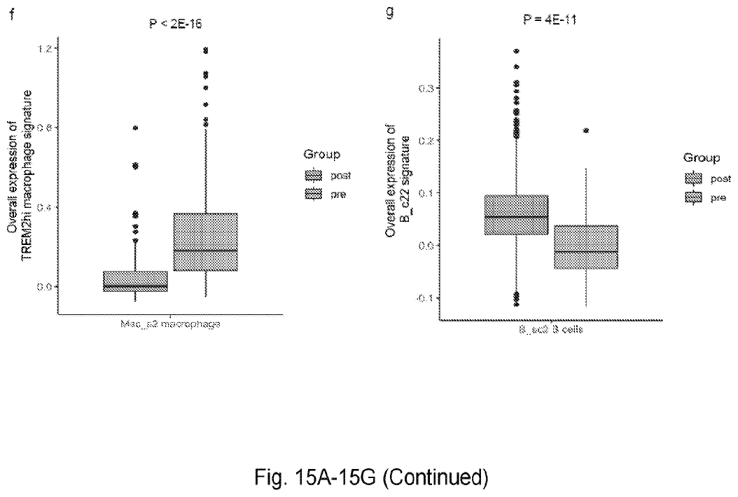

We also analyzed a single-cell RNA-seq dataset of basal cell carcinoma (BCC) patients before and after anti-PD-1 therapy⁸. We found that the results of our study can be generalized to BCC treated with ICT. Although this BCC scRNA-seq dataset did not sequence the γδ T cells, the results for macrophages and B cells in this BCC dataset are similar to our findings for the melanoma dataset. First, we did general clustering analyses and identified the overall macrophages and B cells populations (FIG. 15a). Then we performed finer clustering and identified the macrophages and B-cell subpopulations from the BCC tumors that are similar to the TREM2^{hi} macrophages and B_c22 B cells in the initial melanoma samples (FIG. 15b-e). In the BCC dataset, the Mac_s2 macrophage subcluster overexpressed the TREM2^{hi} macrophage marker genes (TREM2, FOLR2, MMP12, C1QA, C1QB, and C1Qc; FIG. 15d); the B_sc2 B-cell subcluster overexpressed the B_c22 B cells marker genes (TRAC, IL2RA, ITGB1, ZBTB32, TRAF1, and CCND2; FIG. 15e). As before, we validated the overall expression changes of the TREM2^{hi} macrophage signature of the Mac_s2 macrophages and the B_c22 signature of the B_sc2 B cells in response to the anti-PD-1 immunotherapy in this BCC dataset⁸. Specifically, the Mac_s2 macrophage subset had significantly decreased overall expression of the TREM2^{hi} macrophage signature in the responsive BCC tumors after anti-PD-1 therapy when compared to the pretreatment BCC samples (FIG. 15f). The B_sc2 B-cell subset had significantly higher overall expression of the B_c22 signature in the post anti-PD-1 therapy in the responsive BCC tumors than in the pretreatment BCC samples (FIG. 15g). These findings suggest that the immune cell subpopulations that we had identified as associated with outcomes of cancer immunotherapy for melanoma also exist in BCC, and that the characteristic gene expression signatures may be altered similarly in melanoma and in BCC in the context of response to immunotherapy.

The Development of an ICT Outcome Signature

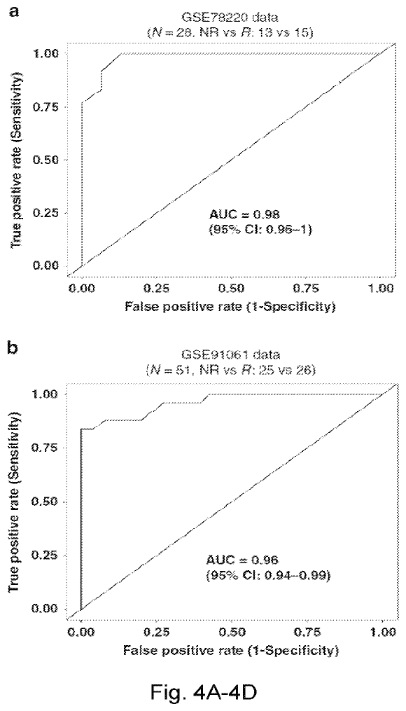

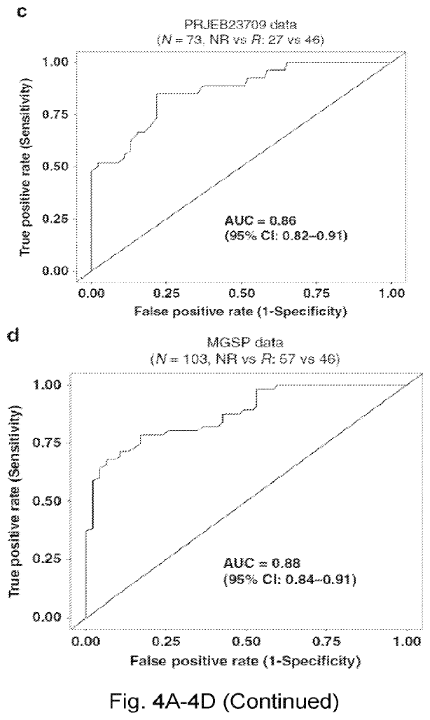

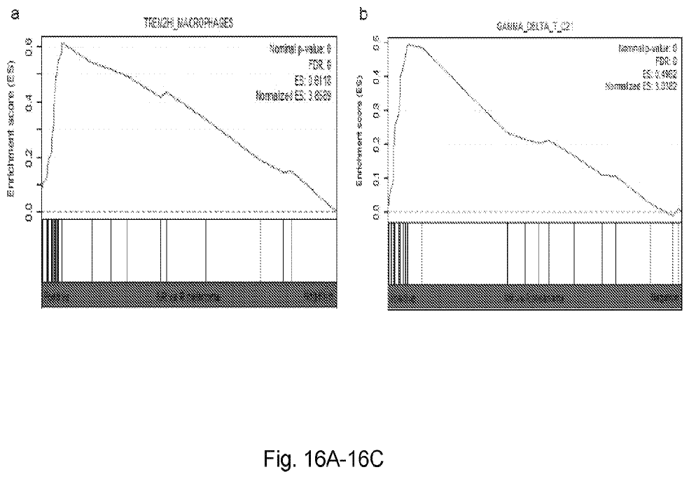

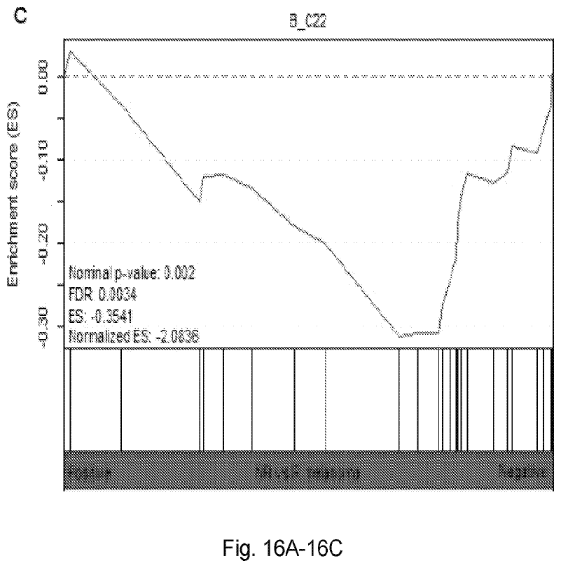

Because the TREM2^{hi} Mφ, Tgd_c21 and B_c22 populations exhibited the greatest quantitative differences between ICT non-responders and responders, we hypothesized that the expression of the feature genes of these populations may predict ICT outcome. To explore this hypothesis, we developed an ICT responsiveness signature based on the scRNA-seq dataset and a bulk gene expression dataset—GSE78220⁹ using the cancerclass R package²³. This signature had significantly high prognostic values for ICT outcomes in the discovery dataset. Specifically, for the GSE78220⁹ dataset (N=28, NR vs R: 13 vs 15), the signature had an AUC (Area Under The Curve) of 0.98 (95% confidence interval [CI], 0.96-1), sensitivity of 93% (95% CI, 72-100%), and specificity of 85% (95% CI, 59-97%; FIG. 4a). In the GSE78220 dataset, only one sample was early-on-treatment tumor and all the rest 27 melanoma samples are from pretreatment tumors. Because this ICT outcome signature was enriched for the characteristic genes of TREM2^{hi} Mφ, Tgd_c21, B_c22 immune cell subpopulations (FIG. 16), it was named as ImmuneCells.Sig. Detailed information of the genes of this signature can be found in Table 1. Then the performance of ImmuneCells.Sig to predict ICT outcome was further validated using multiple independent bulk gene expression datasets of the pretreatment samples as follows.

To validate the above ICT response signature—ImmuneCells.Sig, we analyzed three independent gene expression datasets of melanoma patients to test the predictive performance of ImmuneCells.Sig^{10_12}. For the first two datasets (GSE91061 and PRJEB23709)^{10,11}, the pretreatment melanoma samples were selected for validation. Neither of these datasets were used to develop the ImmuneCells.Sig. For the GSE91061 dataset (N=51, NR vs R: 25 vs 26), ImmuneCells.Sig performed well in differentiating NR from R tumors with an AUC of 0.96 (95% CI, 0.94-0.99), sensitivity of 88% (95% CI, 72-97%), and specificity of 92% (95% CI, 78-99%; FIG. 4b). For the PRJEB23709 dataset (N=73, NR vs R: 27 vs 46), ImmuneCells.Sig also accurately predicted ICT outcomes: AUC of 0.86 (95% confidence interval [CI], 0.82-0.91), sensitivity of 78% (95% CI, 61-90%), and specificity of 78% (95% CI, 66-88%; FIG. 4c). The binomial confidence intervals for sensitivity and specificity were calculated by the Wilson procedure implemented in the cancerclass R package²³.

For further validation, we downloaded and analyzed the third dataset that includes the gene expression profile of a big cohort of melanoma patients who were treated by the anti-PD-1 immunotherapy, from which a large number of pretreatment melanoma samples from 103 patients with distinct response to ICT (46 responders vs 57 non-responders) had been subjected to RNA-seq¹². Applied to this large dataset that was named as MGSP (melanoma genome sequencing project), the predictive value of ImmuneCells.Sig was still high. Specifically, it differentiated progressors from responders with an AUC of 0.88 (95% CI, 0.84-0.91), sensitivity of 79% (95% CI, 68-87%), and specificity of 79% (95% CI, 67-88%; FIG. 4d).

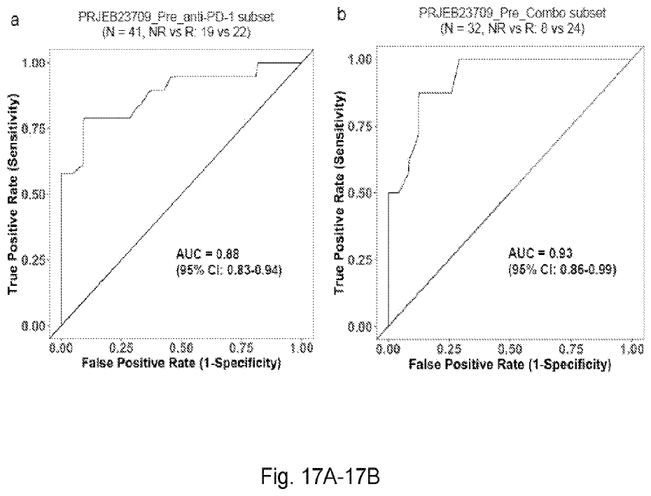

Among the four bulk RNA-seq datasets, only the PRJEB23709 dataset had pre-ICT biopsies for melanoma patients treated with either anti-PD-1 (41 patients: 19 non-responders vs 22 responders) or the combination of anti-PD-1 and anti-CTLA-4 drugs (32 patients: 8 non-responders vs 24 responders). We split the PRJEB23709 dataset into PRJEB23709_Pre_anti-PD-1 and PRJEB23709_Pre_Combo according to the treatment scheme (anti-PD-1 or combination of anti PD-1 and anti-CTLA-4). In each dataset, we tested the performance of ImmuneCells.Sig. It was found that ImmuneCells.Sig can accurately distinguish responders from non-responders in both Pre_anti-PD-1 and Pre_Combo subgroups. For PRJEB23709_Pre_anti-PD-1 subset, the performance of ImmuneCells.Sig is as follows: AUC=0.88 (95% CI, 0.83-0.94), sensitivity=86% (95% CI, 68-96%), and specificity=79% (95% CI, 58-92%; FIG. 17a). For PRJEB23709_Pre_Combo subset, the performance of ImmuneCells.Sig is as follows: AUC=0.93 (95% CI, 0.86-0.99), sensitivity=88% (95% CI, 71-97%), and specificity=88% (95% CI, 53-99%; FIG. 17b).

Using the R package cancerclass, we can calculate the z-score in each pre-therapy biopsy based on the expression values of the ImmuneCells.Sig genes to predict who are more likely to respond to anti-PD-1 or anti-PD-1 plus anti-CTLA-4 combo therapy. For example, in the model built from Pre-anti-PD-1 dataset of PRJEB23709_Pre_anti-PD-1, the threshold z-score of 0.19 yielded sensitivity of 91% for responders. In the model built from Pre-Combo dataset of PRJEB23709_Pre_Combo, the threshold z-score of 0.1 yielded sensitivity of 91% for responders. Therefore, if we test a pre-therapy melanoma sample, the corresponding patient may not respond to either anti-PD-1 treatment or anti-PD-1 plus anti-CTLA-4 combo treatment if the z-score is <0.1, but may respond to the more toxic combo treatment if z-score is within the range of [0.1, 0.19], and may respond to the less toxic anti-PD-1 treatment alone if the z-score is >0.19. Therefore, prediction of the outcomes of different therapy regimen is possible based on the application of ImmuneCells.Sig.

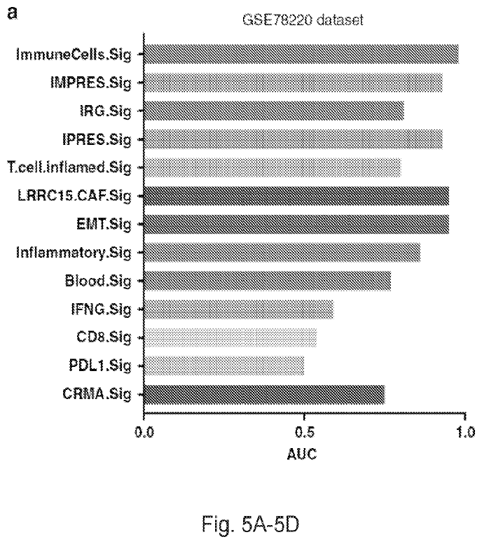

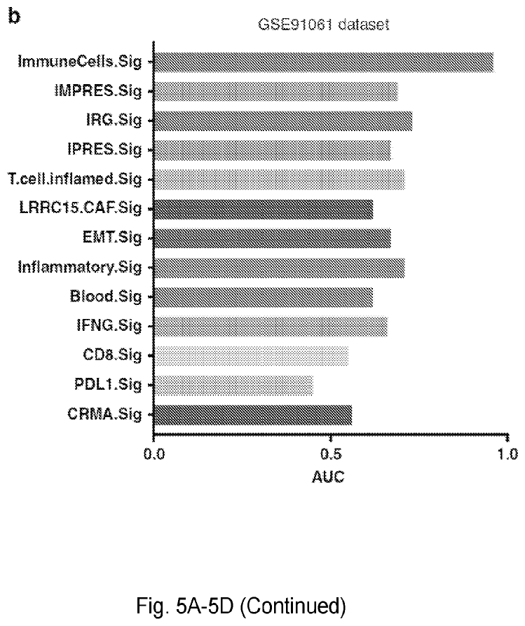

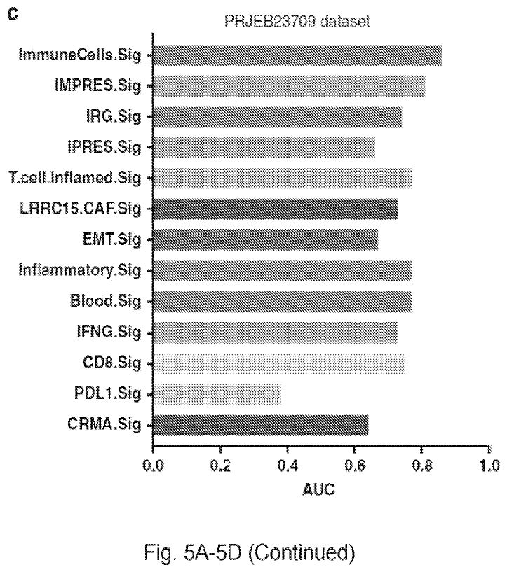

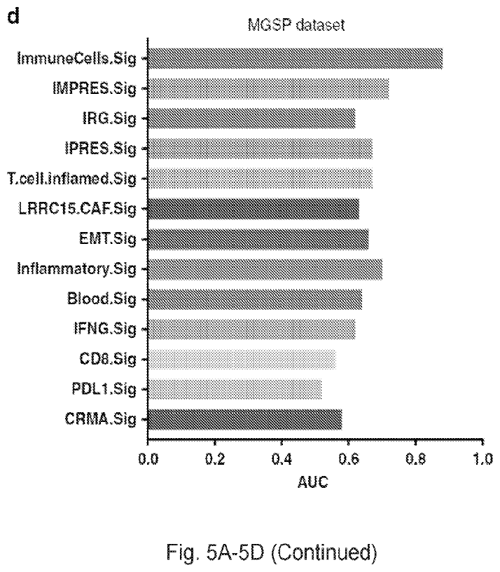

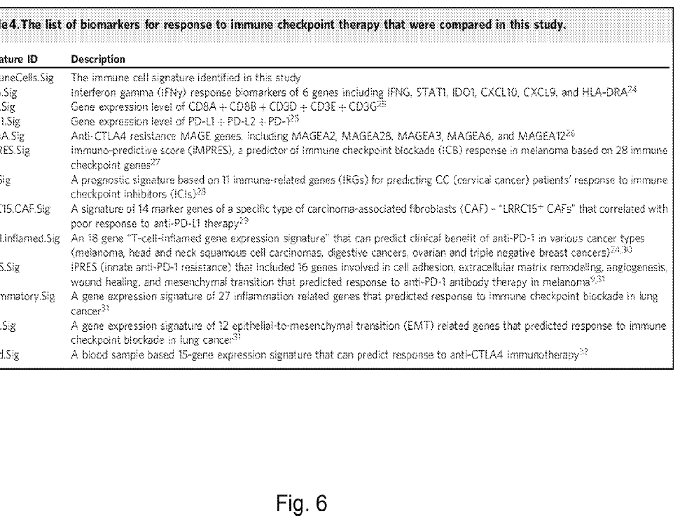

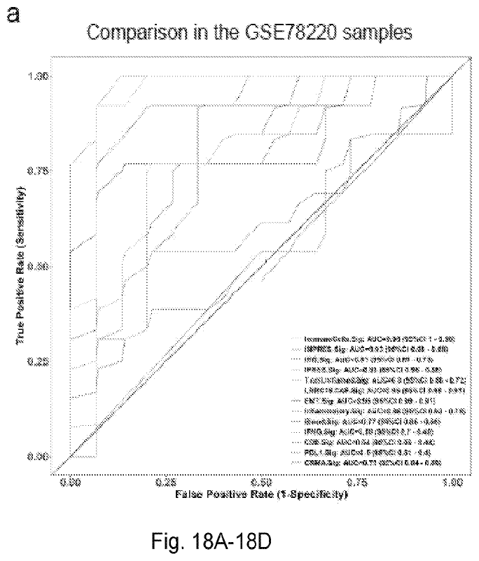

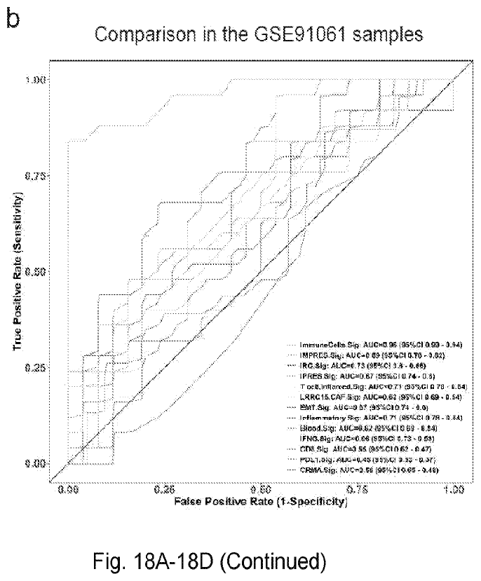

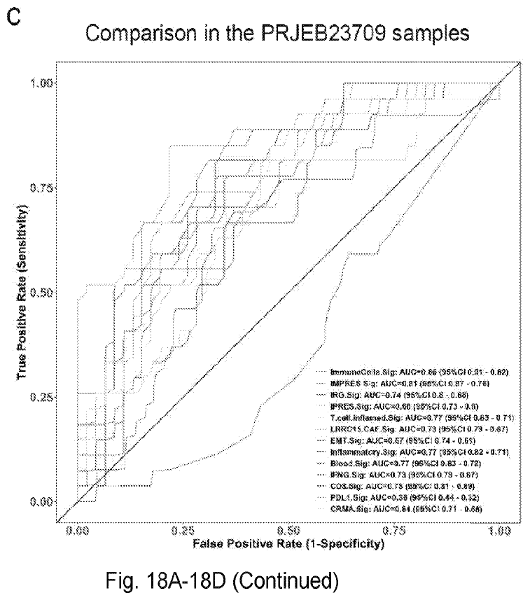

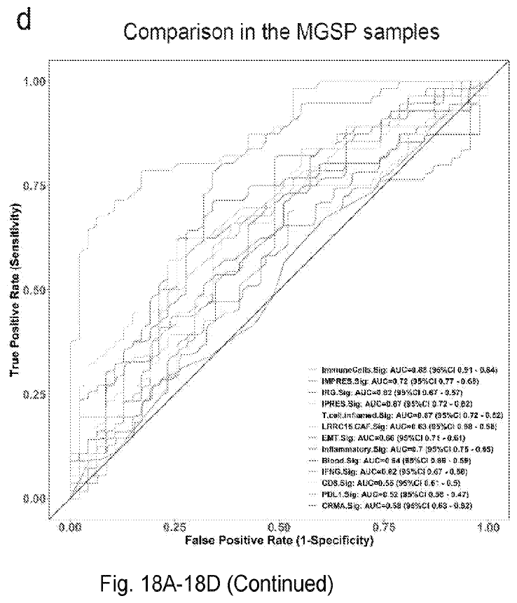

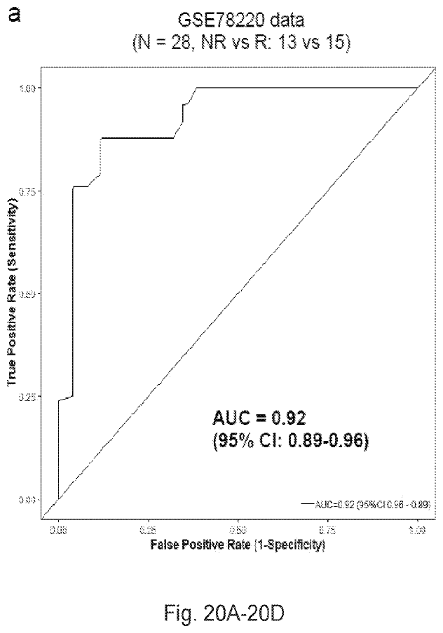

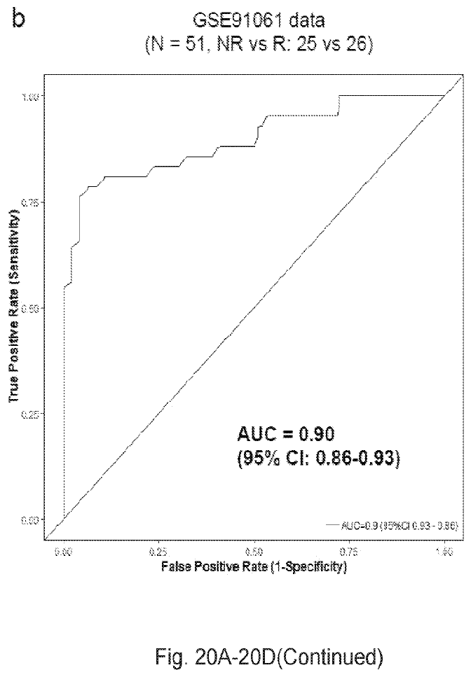

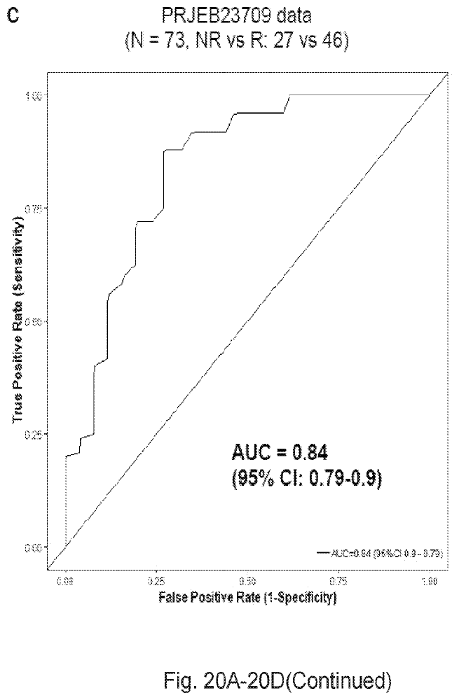

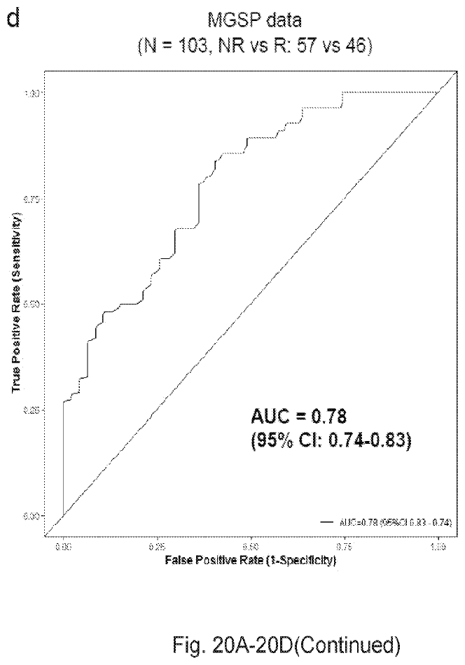

To further evaluate the predictive performance of the ImmuneCells.Sig signature, we compared the ImmuneCells.Sig with the other 12 ICT response signatures reported previously (FIG. 6)^{9,24_32}, including the previously recognized IMPRES signature²⁷; they were all compared across the above four transcriptome-wide gene expression datasets of melanoma patients (i.e., the GSE78220, GSE91061, PRJEB23709, and MGSP datasets). The results show that the ImmuneCells.Sig was consistently the best signature for predicting response to immunotherapy across all four datasets (FIG. 5 and FIG. 18). As a reference, the well-established IMPRES signature was ranked third in prediction accuracy in the GSE78220 dataset (FIG. 5a and FIG. 18a), fifth in the GSE91061 dataset (FIG. 5b and FIG. 18b), and second in both the PRJEB23709 and the MGSP datasets (FIG. 5c, d and FIG. 20c, d). The fact that the ImmuneCells.Sig signature is the best predictor for the outcome of immune checkpoint therapy across the four independent melanoma datasets suggests that the ImmuneCells.Sig is an effective biomarker that can accurately predict ICT clinical outcome based on the pretreatment tumor samples from melanoma patients.

### Discussion

A large-scale single-cell RNA-seq study of tumor samples of melanoma patients treated by ICT⁶ was re-analyzed to dissect individual cell populations that may correlate with response. Three immune cell clusters had drastically different percentages in ICT responders vs non-responders. The TREM2^{hi} macrophages and Tgd_c21 T cells were markedly higher in the non-responders and could contribute to ICT resistance; in contrast, the B_c22 B cells were higher in the responders and could contribute to ICT anti-tumor response. TREM2^{hi} macrophages, the most enriched immune cell subcluster in the non-responders, displayed a distinct gene expression pattern, with overexpression of key genes of the complement system. Expression of complement effectors and receptors has been associated with cancer progression and poor prognosis^{33,34}. Among all the complement elements that may have the pro-cancer activities, C1q chains, C3-derived fragments, and C5a are likely the most important modulators of tumor progression^{35,36}. In a clear-cell renal cell carcinoma (ccRCC) model, mice deficient in C1q, C4, or C3 displayed decreased tumor growth, whereas tumors infiltrated with high densities of C1q-producing macrophages exhibited an immunosuppressed microenvironment³⁷. The classical complement pathway is a key inflammatory mechanism that is activated by cooperation between tumor cells and tumor-associated macrophages, favoring cancer progression³⁷. Our findings extend this premise; TREM2^{hi} macrophages, which overexpress major elements of the complement system and activation of the complement cascade, are enriched in ICT non-responders and could be the major macrophage subset that contributes to ICT resistance.

Although the role of complement system is not completely understood, other studies described different mechanisms by which complement activation in the tumor microenvironment can enhance tumor growth, such as altering the immune profile of tumor-infiltrating leukocytes, increasing cancer cell proliferation, and suppressing CD8+ TIL function³⁸. More recently, complement effectors such as C1q, C3a, C5a, and others have been associated with inhibition of anti-tumor T-cell responses through the recruitment and/or activation of immunosuppressive cell subpopulations such as MDSCs (myeloid-derived suppressor cells), Tregs, or M2 tumor-associated macrophages (TAMs)³⁹. The rationale of inhibiting the complement system for therapeutic combinations to enhance the anti-tumor efficacy of anti-PD-1/PD-L1 checkpoint inhibitors has been proposed based on the supporting evidence that complement blocks many of the effector routes associated with the cancer-immunity cycle³⁹. Our study results were in line with these findings and suggest that the TREM2^{hi} macrophage population which has an activated complement system could be another source or consequence of complement activation contributing to the blockade of cancer-immunity cycle.

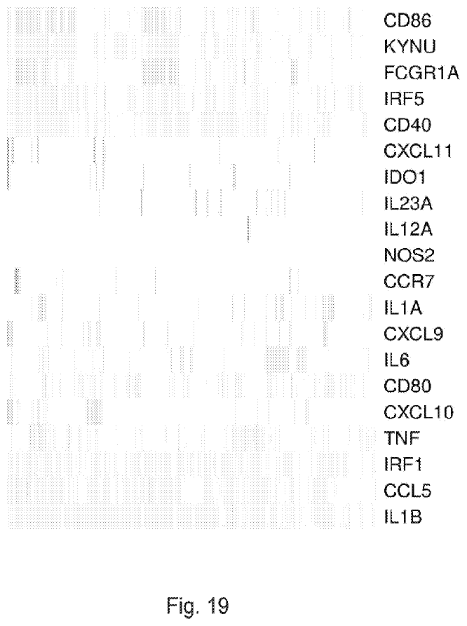

Many M2 polarization genes, some of which are known to be tumor-promoting, were also overexpressed in TREM2^{hi} macrophages. For example, CD276 (B7-H3) plays a role in down-regulating T-cells involved in tumor immunity^{40,41}. High CD276 expression is associated with increased tumor size, lymphovascular invasion, poorly differentiated tumors, and shorter overall patient survival^{42,43}. CD276 expression is also associated with tumor-infiltrating FOXP3+ regulatory T cells which inhibit effector T cells^{44,45} and is important for immune evasion and tumorigenesis in prostate cancer⁴⁶. CD276 also inhibits NK cell lysis of tumor cells⁴⁷. The overexpression of CD276 in TREM2^{hi} macrophages likely has implications for promoting ICT resistance. PD-L2, an important immune co-inhibitory molecule⁴⁸, was also overexpressed in the TREM2^{hi} macrophages. Increased expression of PD-L2 in tumor-associated macrophages contributes to suppressing anti-tumor immunity in mice treated with anti-PD-L1 monoclonal antibody⁴⁹. Thus, the high PD-L2 expression in TREM2^{hi} macrophages could facilitate ICT resistance and tumor progression. Some single-cell studies reported that M1 and M2 signatures are positively correlated in myeloid populations^{50,51}. We checked the expression of M1 markers from these studies in the TREM2^{hi} macrophages (FIG. 19). It was found that the expression of M1 signature genes was neither strong nor consistent. The gene—iNOS (NOS2), the most characteristic and canonical M1 macrophage marker^{20,50_52} was not expressed in the TREM2^{hi} cell population (FIG. 19). These results suggest that TREM2^{hi} macrophages are functionally more proximal to M2 polarization macrophages. TREM2^{hi} macrophages had been reported in a breast cancer single-cell study to be a branch of recruited or resident M2 type macrophages expressing several genes in common with our study such as SSP1, C1Q, CCL18, and MACR050. However, TREM2^{hi} macrophages had not been linked to cancer immunotherapy response before. So that aspect of our data is valuable to clinical practice in cancer immunotherapy.

A γδ T cells subset, Tgd_c21, was present at much higher levels in the non-responders. Despite their role in anti-tumor cytotoxicity, γδ T cells could also promote cancer progression by inhibiting anti-tumor responses and enhancing cancer angiogenesis. Consequently, γδ T cells have a dual effect and are considered as being both friends and foes of cancer⁵³. The enrichment of the Tgd_c21 cells in the ICT non-responders suggests an association with ICT resistance. The top Tgd_c21 marker genes are oncogenic by nature including RM2⁵⁴, BIRC5 (Survivin)⁵⁵, SPC24^{56,57}, UBE2C^{58,59}, and CDCA5⁶⁰. Pathway analysis revealed a significant reduction in ligand-receptor binding capacity, IFNα and IFNβ signaling, IFN-γ response, and immunoregulatory interactions of Tgd_c21 cells, suggesting that Tgd_c21 cells may be a type of ‘exhausted’ γδ T cell with impaired anti-tumor immune functions. A previous study showed that the positive outcome of PD-1 blockade on treating leukemia may be because that it induces significant upregulation of the potent pro-inflammatory and anti-tumor cytokine IFN-γ in certain types of γδ T cells⁶¹. Complementing their study, we showed that the failure of immunotherapy in treating melanoma may be associated with some types of γδ T cells (e.g., Tgd_c21). The pathway analysis showed that this subset of γδ T cells—Tgd_c21 had decreased activity of the anti-tumor IFN-γ pathway in the non-responders than the responders subjected to the immunotherapy (FIG. 3c). Therefore, a key element may be the IFN-γ pathway activity, whose reduction in some γδ T-cell subsets such as Tgd_c21 in ICT non-responders may contribute to ICT resistance.

All B-cell clusters were depressed in the ICT non-responders. Apart from their role in antibody production, B cells also are an important source of cytokines and chemokines that contribute to anti-tumor immune responses⁶². Therefore, the decreased B-cell percentages in non-responders could contribute to ICT resistance and/or progression of ICT-resistant tumors. We compared the present B-cell subpopulation signature (B_c22, based on cutoff P value 0.05) with the other B-cell signature recently published in the context of ICT by Helmink et al.⁶³ and found several genes shared by both signatures including TCL1A, ITIH5, LAX1, KCNA3, CD79A, AREG, GBP1, ATP8A, and IGLL5. Both our signature and their signature characterized the B-cell populations that were significantly enriched in the ICT responders versus non-responders. However, the B cells associated with these two signatures were different. This is because our B_c22 (single cell cluster 22) signature was developed based on the scRNA-seq data of melanoma samples and its corresponding B cells were a subset of B cells that were highly enriched in the ICT responders than the non-responders. We also identified three other B-cell subpopulations corresponding to clusters 13, 14, and 17 (FIG. 1 and FIG. 21). In contrast, the B-cell signature used by Helmink et al.⁶³ was derived from bulk RNA-seq data of renal cell carcinoma (RCC); thus, their signature may represent a mix of B-cell subpopulations enriched in RCC patients that responded to ICT. Therefore, it is logical for the two signatures to share some, but not all genes.

For comparison with ImmuneCells.Sig, we used the gene signature representing the three component cell clusters (TREM2^{hi} macrophages, Tgd_c21 γδ T cells, and B_c22 B cells) identified from the scRNA-seq data (FIG. 2 and FIG. 14). This 150-gene signature is composed of three sets of top 50 genes most significantly overexpressed in one of the three cell clusters. This gene signature was called scR.Immune and used for ICT outcome prediction. The scR.Immune signature had a somewhat lower predictive capability compared with the ImmuneCells.Sig signature derived from both scRNA-seq and bulk gene expression datasets. As seen in FIG. 21, the AUC values from scR.Immune were 0.92, 0.90, 0.84, and 0.78 for the datasets of GSE78220, GSE91061, PRJEB23709, and MGSP, respectively, which are lower than the AUC values given by the ImmuneCells.Sig signature (0.98, 0.96, 0.86, and 0.88 for the four datasets, respectively). The difference in predictability between these two sets of signatures is likely due to the complex cellular composition of tumors. Because the four datasets used for AUC calculations are all bulk gene expression data, the corresponding expression levels of genes represented a mix of expression from all kinds of cells embedded in the tumor samples. Therefore, using scRNA-seq data derived signature alone such as the scR.Immune signature may not predict ICT outcome better than using the ImmuneCells.Sig signature derived from both scRNA-seq and bulk gene expression datasets. However, the ImmuneCells.Sig signature is enriched for the signature genes from the TREM2^{hi} macrophages, Tgd_c21 γδ T cells and B_c22 B cells, suggesting the involvement of these immune cell subpopulations in determining the ICT responsiveness. This signature may also be useful to predict progressive versus responsive melanoma tumors extracted from the same patients treated with ICT⁶⁴. A limitation of this study is that deciphering the biological meanings of the above relevant cell types that impact the efficacy of ICT treatment remains unsolved. Well-designed experimental strategies should be used to explore the hidden mechanisms to strengthen the biological findings of this study.

The decreased percentage of B cells and increased percentage of macrophages/monocytes in ICT non-responding patients had been reported previously⁶. However, the important subsets of these immune cell populations were not revealed as in this study. Moreover, we identified an ICT outcome gene expression signature, ImmuneCells.Sig, that is enriched for the characteristic genes of TREM2^{hi} macrophages, Tgd_c21, and B_c22 subpopulations. The ImmuneCells.Sig signature outperformed the other outstanding signatures in predicting the outcome of immune checkpoint therapies across all four independent datasets^{9_12}. Our characterization of these immune cell populations provides the opportunities to improve the efficacy of cancer immunotherapy and to better understand the mechanisms of ICT resistance.

### Methods

Study Design

Single-cell RNA-sequencing data (accession number GEO: GSE120575) of melanoma samples from the initial publication⁶ were down-loaded and re-analyzed for this manuscript. For the validation purposes, two other scRNA-seq datasets^{7,8} of melanoma and BCC were also downloaded, which are accessible through GEO accession number: GSE115978 and GSE123813. For the development of the ICT outcome signature, we analyzed the transcriptome-level gene expression data set (GSE78220) of an immune checkpoint therapy (ICT) study⁹. For the validation of the identified ICT outcome signature—ImmuneCells.Sig, we analyzed three large public gene expression datasets of immunotherapy^{10_12} (respectively accession number: GSE91061, ENA project PRJEB23709, dbGaP phs000452.v3.p1). The first dataset¹⁰ (GSE91061) consisted of pretreatment melanoma samples from 51 patients (25 non-responders and 26 responders). For the second dataset¹¹ (PRJEB23709), the scRNA-seq data of the 73 pretreatment tumors were analyzed. Among these 73 samples, 41 are from the melanoma patients subjected to anti-PD-1 therapy and consist of 19 non-responders and 22 responders; 32 are from the melanoma patients subjected to combined anti-PD-1 and anti-CTLA-4 therapy and consist of 8 non-responders and 24 responders. The third dataset (phs000452.v3.p1) is from a large melanoma genome sequencing project (MGSP)¹² from which the whole-transcriptome sequencing (RNA-seq) data from 103 pretreatment tumor tissue samples from 103 patients with distinct ICT outcomes (47 responders and 56 non-responders) were available and used for validation in this study.

Single-Cell RNA Sequencing Data Analysis

The data from a previous scRNA-seq study of melanoma checkpoint immunotherapy⁶ were analyzed. Specifically, we utilized the Seurat v3.0 R package^{13,14} to perform the fine clustering of the 16,291 single cells. The gene expression data from single cells of both conditions, i.e., regression/responder (R group: n.patients=17; n.cells=5564) and progression/non-responders (NR group: n.patients=31; n.cells=10,727), were aligned and projected in a 2-dimensional space through uniform manifold approximation and projection (UMAP)¹⁶ to allow identification of ICT-outcome-associated immune cell populations. Highly variable genes—genes with relatively high average expression and variability—were detected with Seurat^{13,14}. These genes were used for downstream clustering analysis. Principal component analysis (PCA) was used for dimensionality reduction and the number of significant principal components was calculated using built in the JackStraw function. t-distributed stochastic neighbor embedding (t-SNE) and UMAP were used for data visualization in two dimensions.

The built-in FindMarkers function in the Seurat package was used to identify differentially expressed genes. From the results of the Seurat package, genes with adjusted P values<0.05 were considered as differentially expressed genes. Adjusted P values were calculated based on Bonferroni correction using all features in the dataset following Seurat manual [https://satijalab.org/seurat/v3.0/de_vignette.html]. Genes retrieved from Seurat analysis were displayed in heatmap using scaled gene expression calculated with the Seurat-package built-in function. Fold change plots were created in R with ggplot2 package. For the two scRNA-seq data^{7,8} of melanoma and BCC that were used for validation, i.e., GSE115978 and GSE123813 datasets, the pre-processed gene expression data were downloaded, processed, and analyzed in the same way as done for the discovery scRNA-seq dataset—GSE120575.

RNA-Seq Data and ICT Responsiveness Signature Analysis

For the bulk RNA-seq datasets^{9_11}, we processed them in the following steps. The downloaded FASTQ files containing the RNA-seq reads were aligned to the hg19 human genome using Bowtie-TopHat (version 2.0.4)^{65,66}. Gene-level read counts were obtained using the htseq-count Python script from HTSeq v0.11.1 [https://htseq.readthedocs.io/en/release_0.11.1/] in the union mode. We further utilized the iDEP v0.9267 [http://bioinformatics.sdstate.edu/idep/] to transform the read counts data using the regularized log (rlog) transformation method originally implemented in the DESeq2 v1.28.1 package⁶⁸ [https://bioconductor.org/packages/release/bioc/html/DESeq2.html], as it effectively reduces mean-dependent variance. The transformed data are used for the downstream analysis and available as detailed in the Data availability statement.

Because three single-cell clusters—TREM2^{hi} macrophages, Tgd_c21, and B_c22 exhibited large quantitative changes between the ICT responders and non-responders, we hypothesized that the tumor expression of the feature genes of these specific immune cell populations may be useful to predict the ICT outcome. In order to test this hypothesis, we developed an ICT responsiveness signature based on the scRNA-seq dataset and a bulk gene expression dataset—GSE78220⁹ using the cancerclass R package²³. To validate this ICT response signature—ImmuneCells.Sig, we analyzed three independent gene expression datasets of melanoma patients^{10_12} (GSE91061, PRJEB23709, and MGSP datasets) and corroborated the high prediction values of ImmuneCells.Sig. We also compared the ImmuneCells.Sig with the other 12 ICT response signatures reported previously (Table 1)^{9,24_32} across the above four gene expression datasets of melanoma patients. The corresponding R codes are available as detailed in the Code availability statement.

Pathway Analyses

Pathway analyses were conducted using several excellent software tools, including IPA software (IPA release June 2020, QIAGEN Inc., [https://www.qiagenbioinformatics.com/products/ingenuitypathway-analysis]), Gene Set Variation Analysis⁶⁹ (GSVA v1.36.2, [https://bioconductor.org/packages/release/bioc/html/GSVA.html]), and Gene Set Enrichment Analysis²² (GSEA v4.0.0, [https://www.gsea-msigdb.org/gsea/index.jsp]). GSEA analysis was performed for pre-ranked differentially expressed genes using the option—GseaPreranked. One thousand permutations were used to calculate significance. A gene set was considered to be significantly enriched in one of the two groups when the raw P value<0.05 and the FDR (false discovery rate) was <0.25 for the corresponding gene set. In addition, we utilized an R-package called Fast Gene Set Enrichment Analysis (fgsea v1.15.1, [https://github.com/ctlab/fgsea]). The package implements a special algorithm to calculate the empirical enrichment score null distributions simultaneously for all the gene set sizes, which allows up to several hundred times faster execution time compared to original Broad implementation of GSEA. Reactome pathways analyses were performed using Protein ANalysis THrough Evolutionary Relationships (PANTHER v15.0, [http://pantherdb.org/]). The associated settings are—Analyze type: PANTHER Overrepresentation Test, release 20190711; Annotation Version and Release Date: Gene Ontology database Released 2019-07-03 [http://geneontology.org/]) with lists of significantly enriched genes in the corresponding clusters as detected by Seurat.

Statistical Analysis

The performance of the ImmuneCells.Sig as a classifier for ICT outcome was evaluated with the use of receiver-operating-characteristic curves (ROC), calculation of AUC (Area under the ROC Curve), and estimates of sensitivity and specificity implemented in the cancerclass v1.32.0 R package²³. This classification protocol starts with a feature selection step and continues with nearest-centroid classification. The binomial confidence intervals for sensitivity and specificity were calculated by the Wilson procedure implemented in the cancerclass R package²³. Fisher's exact test was used for categorical variables. All confidence intervals are reported as two-sided binomial 95% confidence intervals. Statistical analysis was performed with R software, version 3.5.3 (R Project for Statistical Computing).

### REFERENCES

1. Larkin J, et al. Combined nivolumab and ipilimumab or monotherapy in untreated melanoma. New Engl. J. Med. 2015; 373:23-34.
2. Rizvi N A, et al. Cancer immunology. Mutational landscape determines sensitivity to PD-1 blockade in non-small cell lung cancer. Science. 2015; 348:124-128.
3. Tumeh P C, et al. PD-1 blockade induces responses by inhibiting adaptive immune resistance. Nature. 2014; 515:568-571.
4. Sade-Feldman M, et al. Resistance to checkpoint blockade therapy through inactivation of antigen presentation. Nat. Commun. 2017; 8:1136.
5. Gao J, et al. Loss of IFN-gamma pathway genes in tumor cells as a mechanism of resistance to anti-CTLA-4 therapy. Cell. 2016; 167:397-404.e399.
6. Sade-Feldman M, et al. Defining T cell states associated with response to checkpoint immunotherapy in melanoma. Cell. 2018; 175:998-1013.e1020.
7. Jerby-Arnon L, et al. A cancer cell program promotes T cell exclusion and resistance to checkpoint blockade. Cell. 2018; 175:984-997 e924.
8. Yost K E, et al. Clonal replacement of tumor-specific T cells following PD-1 blockade. Nat. Med. 2019; 25:1251-1259.
9. Hugo W, et al. Genomic and transcriptomic features of response to anti-PD-1 therapy in metastatic melanoma. Cell. 2016; 165:35-44. doi: 10.1016/j.cell.2016.02.065.
10. Riaz N, et al. Tumor and microenvironment evolution during immunotherapy with nivolumab. Cell. 2017; 171:934-949.e915.
11. Gide T N, et al. Distinct immune cell populations define response to anti-PD-1 monotherapy and anti-PD-1/anti-CTLA-4 combined therapy. Cancer Cell. 2019; 35:238-255 e236.
12. Liu D, et al. Integrative molecular and clinical modeling of clinical outcomes to PD1 blockade in patients with metastatic melanoma. Nat. Med. 2019; 25:1916-1927.
13. Butler A, Hoffman P, Smibert P, Papalexi E, Satija R. Integrating single-cell transcriptomic data across different conditions, technologies, and species. Nat. Biotechnol. 2018; 36:411-420.
14. Stuart T, et al. Comprehensive integration of single-cell data. Cell. 2019; 177:1888-1902.e1821.
15. Eisenhauer E A, et al. New response evaluation criteria in solid tumours: revised RECIST guideline (version 1.1) Eur. J. Cancer. 2009; 45:228-247.
16. Becht, E. et al. Dimensionality reduction for visualizing single-cell data using UMAP. Nat. Biotechnol.37, 38-44 (2019).
17. Pizzolato G, et al. Single-cell RNA sequencing unveils the shared and the distinct cytotoxic hallmarks of human TCRVdelta1 and TCRVdelta2 gammadelta T lymphocytes. Proc. Natl Acad. Sci. USA. 2019; 116:11906-11915.
18. Pont F, Tosolini M, Fournie J J. Single-Cell Signature Explorer for comprehensive visualization of single cell signatures across scRNA-seq datasets. Nucleic Acids Res. 2019; 47:e133.
19. Butte, A. J. et al. Determining significant fold differences in gene expression analysis. Pac. Symp. Biocomput.6, 6-17 (2001).
20. Gubin M M, et al. High-dimensional analysis delineates myeloid and lymphoid compartment remodeling during successful immune-checkpoint cancer therapy. Cell. 2018; 175:1014-1030.e1019.
21. Mi H, Muruganujan A, Thomas P D. PANTHER in 2013: modeling the evolution of gene function, and other gene attributes, in the context of phylogenetic trees. Nucleic Acids Res. 2013; 41:D377-D386.
22. Subramanian A, et al. Gene set enrichment analysis: a knowledge-based approach for interpreting genome-wide expression profiles. Proc. Natl Acad. Sci. USA. 2005; 102:15545-15550.
23. Budczies J, et al. cancerclass: an R Package for development and validation of diagnostic tests from high-dimensional molecular data. J. Stat. Softw. 2014; 59:1-19.
24. Ayers M, et al. IFN-gamma-related mRNA profile predicts clinical response to PD-1 blockade. J. Clin. Invest. 2017; 127:2930-2940.
25. Jiang P, et al. Signatures of T cell dysfunction and exclusion predict cancer immunotherapy response. Nat. Med. 2018; 24:1550-1558.
26. Shukla S A, et al. Cancer-germline antigen expression discriminates clinical outcome to CTLA-4 blockade. Cell. 2018; 173:624-633.e628.
27. Auslander N, et al. Robust prediction of response to immune checkpoint blockade therapy in metastatic melanoma. Nat. Med. 2018; 24:1545-1549.
28. Yang S, et al. Identification of a prognostic immune signature for cervical cancer to predict survival and response to immune checkpoint inhibitors. Oncoimmunology. 2019; 8:e1659094.
29. Dominguez, C. X. et al. Single-cell RNA sequencing reveals stromal evolution into LRRC15+ myofibroblasts as a determinant of patient response to cancer immunotherapy. Cancer Discov.10, CD-19-0644 (2019).
30. Huang A C, et al. A single dose of neoadjuvant PD-1 blockade predicts clinical outcomes in resectable melanoma. Nat. Med. 2019; 25:454-461.
31. Thompson J C, et al. Gene signatures of tumor inflammation and epithelial-to-mesenchymal transition (EMT) predict responses to immune checkpoint blockade in lung cancer with high accuracy. Lung Cancer. 2020; 139:1-8.
32. Friedlander P, et al. Whole-blood RNA transcript-based models can predict clinical response in two large independent clinical studies of patients with advanced melanoma treated with the checkpoint inhibitor, tremelimumab. J. Immunother. Cancer. 2017; 5:67.
33. Imamura T, et al. Influence of the C5a-05a receptor system on breast cancer progression and patient prognosis. Breast Cancer. 2016; 23:876-885.
34. Gu J, et al. Overexpression of CD88 predicts poor prognosis in non-small-cell lung cancer. Lung Cancer. 2013; 81:259-265.
35. Bulla R, et al. C1q acts in the tumour microenvironment as a cancer-promoting factor independently of complement activation. Nat. Commun. 2016; 7:10346.
36. Ajona D, Ortiz-Espinosa S, Pio R. Complement anaphylatoxins C3a and C5a: Emerging roles in cancer progression and treatment. Semin. Cell Dev. Biol. 2019; 85:153-163.
37. Roumenina L T, et al. Tumor cells hijack macrophage-produced complement C1q to promote tumor growth. Cancer Immunol. Res. 2019; 7:1091-1105.
38. Afshar-Kharghan V. The role of the complement system in cancer. J. Clin. Invest. 2017; 127:780-789.
39. Pio R, Ajona D, Ortiz-Espinosa S, Mantovani A, Lambris J D. Complementing the cancer-immunity cycle. Front. Immunol. 2019; 10:774.
40. Suh W K, et al. The B7 family member B7-H3 preferentially down-regulates T helper type 1-mediated immune responses. Nat. Immunol. 2003; 4:899-906.
41. Loos M, Hedderich D M, Friess H, Kleeff J. B7-h3 and its role in antitumor immunity. Clin. Dev. Immunol. 2010; 2010:683875.
42. Jin Y, et al. B7-H3 in combination with regulatory T cell is associated with tumor progression in primary human non-small cell lung cancer. Int. J. Clin. Exp. Pathol. 2015; 8:13987-13995.
43. Arigami T, et al. B7-h3 ligand expression by primary breast cancer and associated with regional nodal metastasis. Ann. Surg. 2010; 252:1044-1051.
44. Inamura K, et al. Relationship of B7-H3 expression in tumor cells and tumor vasculature with FOXP3+ regulatory T cells in renal cell carcinoma. Cancer Manag. Res. 2019; 11:7021-7030.
45. Bettelli E, et al. Reciprocal developmental pathways for the generation of pathogenic effector TH17 and regulatory T cells. Nature. 2006; 441:235-238.
46. Zang X, et al. B7-H3 and B7x are highly expressed in human prostate cancer and associated with disease spread and poor outcome. Proc. Natl Acad. Sci. USA. 2007; 104:19458-19463.
47. Cribb G L, Loo S C, Dickinson I. Limb salvage for soft-tissue sarcomas of the foot and ankle. J. Bone Joint Surg. Br. 2010; 92:424-429.
48. Wei S C, Duffy C R, Allison J P. Fundamental mechanisms of immune checkpoint blockade therapy. Cancer Discov. 2018; 8:1069-1086.
49. Umezu D, et al. Inhibitory functions of PD-L1 and PD-L2 in the regulation of anti-tumor immunity in murine tumor microenvironment. Cancer Immunol. Immunother. 2019; 68:201-211.
50. Azizi E, et al. Single-cell map of diverse immune phenotypes in the breast tumor microenvironment. Cell. 2018; 174:1293-1308 e1236.
51. Wagner J, et al. A single-cell atlas of the tumor and immune ecosystem of human breast cancer. Cell. 2019; 177:1330-1345 e1318.
52. Mould, K. J., Jackson, N. D., Henson, P. M., Seibold, M. & Janssen, W. J. Single cell RNA sequencing identifies unique inflammatory airspace macrophage subsets. JCI Insight4, e126556 (2019).
53. Zhao Y, Niu C, Cui J. Gamma-delta (gammadelta) T cells: friend or foe in cancer development? J. Transl. Med. 2018; 16:3.
54. Mazzu Y Z, et al. A novel mechanism driving poor-prognosis prostate cancer: overexpression of the DNA repair gene, ribonucleotide reductase small subunit M2 (RRM2) Clin. Cancer Res. 2019; 25:4480-4492.
55. Li F, Aljandali I, Ling X. Cancer therapeutics using survivin BIRC5 as a target: what can we do after over two decades of study? J. Exp. Clin. Cancer Res. 2019; 38:368.
56. Zhou J, et al. SPC24 Regulates breast cancer progression by PI3K/AKT signaling. Gene. 2018; 675:272-277.
57. Yin H, Meng T, Zhou L, Chen H, Song D. SPC24 is critical for anaplastic thyroid cancer progression. Oncotarget. 2017; 8:21884-21891.
58. Liu G, Zhao J, Pan B, Ma G, Liu L. UBE2C overexpression in melanoma and its essential role in G2/M transition. J. Cancer. 2019; 10:2176-2184.
59. Mo C H, et al. The clinicopathological significance of UBE2C in breast cancer: a study based on immunohistochemistry, microarray and RNA-sequencing data. Cancer Cell Int. 2017; 17:83.
60. Xu J, et al. Systematic cancer-testis gene expression analysis identified CDCA5 as a potential therapeutic target in esophageal squamous cell carcinoma. EBioMedicine. 2019; 46:54-65.
61. Hoeres T, Holzmann E, Smetak M, Birkmann J, Wilhelm M. PD-1 signaling modulates interferon-gamma production by Gamma Delta (gammadelta) T-Cells in response to leukemia. Oncoimmunology. 2019; 8:1550618.
62. Wennhold K, Shimabukuro-Vornhagen A, von Bergwelt-Baildon M. B cell-based cancer immunotherapy. Transfus. Med. Hemother. 2019; 46:36-46.
63. Helmink B A, et al. B cells and tertiary lymphoid structures promote immunotherapy response. Nature. 2020; 577:549-555.
64. Ascierto M L, et al. Transcriptional mechanisms of resistance to anti-PD-1 therapy. Clin. Cancer Res. 2017; 23:3168-3180.
65. Langmead B, Trapnell C, Pop M, Salzberg S L. Ultrafast and memory-efficient alignment of short DNA sequences to the human genome. Genome Biol. 2009; 10:R25.
66. Trapnell C, Pachter L, Salzberg S L. TopHat: discovering splice junctions with RNA-Seq. Bioinformatics. 2009; 25:1105-1111.
67. Ge S X, Son E W, Yao R. iDEP: an integrated web application for differential expression and pathway analysis of RNA-Seq data. BMC Bioinform. 2018; 19:534.
68. Love M I, Huber W, Anders S. Moderated estimation of fold change and dispersion for RNA-seq data with DESeq2. Genome Biol. 2014; 15:550.
69. Hanzelmann S, Castelo R, Guinney J. GSVA: gene set variation analysis for microarray and RNA-seq data. BMC Bioinforma. 2013; 14:7.

TABLE 1
IMMUNECELLS.SIG

| No. | Symbol | Entrez Gene Name | Location | Type(s) | Drug(s) |
|---|---|---|---|---|---|
| 1 | ADAM21 | ADAM metallopeptidase | Plasma | peptidase | |
| | | domain 21 | Membrane | | |
| 2 | ALDH1L2 | aldehyde dehydrogenase 1 | Cytoplasm | enzyme | |
| | | family member L2 | | | |
| 3 | APBB2 | amyloid beta precursor | Cytoplasm | other | |
| | | protein binding family B | | | |
| | | member 2 | | | |
| 4 | APOC2 | apolipoprotein C2 | Extracellular | transporter | |
| | | | Space | | |
| 5 | ARSF | arylsulfatase F | Extracellular | enzyme | |
| | | | Space | | |
| 6 | ASAH2 | N-acylsphingosine | Cytoplasm | enzyme | |
| | | amidohydrolase 2 | | | |
| 7 | ASPM | abnormal spindle | Nucleus | other | |
| | | microtubule assembly | | | |
| 8 | BIRC5 | baculoviral IAP repeat | Cytoplasm | other | gataparsen, EZN 3042 |
| | | containing 5 | | | |
| 9 | C6orf223 | chromosome 6 open | Other | other | |
| | | reading frame 223 | | | |
| 10 | CACNG1 | calcium voltage-gated | Plasma | ion channel | diltiazem/enalapril, |
| | | channel auxiliary subunit | Membrane | | lercanidipin, nitrendipine, |
| | | gamma 1 | | | diltiazem |
| 11 | CCL18 | C-C motif chemokine | Extracellular | cytokine | |
| | | ligand 18 | Space | | |
| 12 | CD177 | CD177 molecule | Cytoplasm | other | |
| 13 | CD244 | CD244 molecule | Plasma | transmembrane | |
| | | | Membrane | receptor | |
| 14 | CDCA5 | cell division cycle | Cytoplasm | other | |
| | | associated 5 | | | |
| 15 | CDH1 | cadherin 1 | Plasma | other | |
| | | | Membrane | | |
| 16 | CEACAM6 | CEA cell adhesion molecule 6 | Plasma | other | L-DOS47 |
| | | | Membrane | | |
| 17 | CILP2 | cartilage intermediate | Extracellular | other | |
| | | layer protein 2 | Space | | |
| 18 | CLDN7 | claudin 7 | Plasma | other | |
| | | | Membrane | | |
| 19 | CLNK | cytokine dependent | Cytoplasm | other | |
| | | hematopoietic cell linker | | | |
| 20 | CORO7 | coronin 7 | Cytoplasm | other | |
| 21 | CR2 | complement C3d receptor 2 | Plasma | transmembrane | |
| | | | Membrane | receptor | |
| 22 | CRTAM | cytotoxic and regulatory T | Plasma | other | |
| | | cell molecule | Membrane | | |
| 23 | CYTL1 | cytokine like 1 | Extracellular | cytokine | |
| | | | Space | | |
| 24 | DHRS9 | dehydrogenase/reductase 9 | Cytoplasm | enzyme | |
| 25 | DLK1 | delta like non-canonical | Extracellular | other | |
| | | Notch ligand 1 | Space | | |
| 26 | DUSP13 | dual specificity | Cytoplasm | phosphatase | |
| | | phosphatase 13 | | | |
| 27 | EIF4A2 | eukaryotic translation | Cytoplasm | translation | |
| | | initiation factor 4A2 | | regulator | |
| 28 | ENTHD1 | ENTH domain containing 1 | Other | other | |
| 29 | FBLN1 | fibulin 1 | Extracellular | other | |
| | | | Space | | |
| 30 | FBLN2 | fibulin 2 | Extracellular | other | |
| | | | Space | | |
| 31 | FMOD | fibromodulin | Extracellular | other | |
| | | | Space | | |
| 32 | FOLR2 | folate receptor beta | Plasma | transporter | |
| | | | Membrane | | |
| 33 | FOXI1 | forkhead box I1 | Nucleus | transcription | |
| | | | | regulator | |
| 34 | GBP1P1 | guanylate binding protein 1 | Other | other | |
| | | pseudogene 1 | | | |
| 35 | GDF1 | growth differentiation | Extracellular | growth factor | |
| | | factor 1 | Space | | |
| 36 | GIMAP4 | GTPase, IMAP family | Nucleus | other | |
| | | member 4 | | | |
| 37 | GPR31 | G protein-coupled receptor 31 | Plasma | G-protein coupled | |
| | | | Membrane | receptor | |
| 38 | GRIA1 | glutamate ionotropic | Plasma | ion channel | methoxyflurane, |
| | | receptor AMPA type | Membrane | | sevoflurane, |
| | | subunit 1 | | | isoflurane |
| 39 | GRM7 | glutamate metabotropic | Plasma | G-protein coupled | fasoracetam |
| | | receptor 7 | Membrane | receptor | |
| 40 | ITGA3 | integrin subunit alpha 3 | Plasma | other | |
| | | | Membrane | | |
| 41 | JMJD7 | jumonji domain containing 7 | Other | other | |
| 42 | KIR2DL4 | killer cell immunoglobulin | Plasma | transmembrane | |
| | | like receptor, two Ig | Membrane | | |
| | | domains and long | | receptor | |
| | | cytoplasmic tail 4 | | | |
| 43 | KIRREL2 | kirre like nephrin family | Plasma | other | |
| | | adhesion molecule 2 | Membrane | | |
| 44 | KLHDC8B | kelch domain containing | Cytoplasm | other | |
| | | 8B | | | |
| 45 | KRT4 | keratin 4 | Cytoplasm | other | |
| 46 | LALBA | lactalbumin alpha | Extracellular | enzyme | |
| | | | Space | | |
| 47 | LEF1 | lymphoid enhancer | Nucleus | transcription | |
| | | binding factor 1 | | regulator | |
| 48 | LINC00243 | long intergenic non- | Other | other | |
| | | protein coding RNA 243 | | | |
| 49 | LYSMD2 | LysM domain containing 2 | Other | other | |
| 50 | MAEL | maelstrom spermatogenic | Nucleus | transcription | |
| | | transposon silencer | | regulator | |
| 51 | MAP2K5 | mitogen-activated protein | Cytoplasm | kinase | |
| | | kinase kinase 5 | | | |
| 52 | MATN3 | matrilin 3 | Extracellular | other | |
| | | | Space | | |
| 53 | MFAP2 | microfibril associated | Extracellular | other | |
| | | protein 2 | Space | | |
| 54 | MKI67 | marker of proliferation Ki-67 | Nucleus | other | |
| 55 | MMP12 | matrix metallopeptidase | Extracellular | peptidase | marimastat |
| | | 12 | Space | | |
| 56 | MMP9 | matrix metallopeptidase 9 | Extracellular | peptidase | glucosamine, |
| | | | Space | | GS-5745 |
| 57 | MT1G | metallothionein 1G | Nucleus | other | |
| 58 | MUSK | muscle associated receptor | Plasma | kinase | |
| | | tyrosine kinase | Membrane | | |
| 59 | MXRA8 | matrix remodeling | Cytoplasm | other | |
| | | associated 8 | | | |
| 60 | MYL1 | myosin light chain 1 | Cytoplasm | other | |
| 61 | MYO1G | myosin IG | Cytoplasm | other | |
| 62 | NACA2 | nascent polypeptide | Other | other | |
| | | associated complex | | | |
| | | subunit alpha 2 | | | |
| 63 | NACA3P | NACA family member 3, | Other | other | |
| | | pseudogene | | | |
| 64 | NUDT10 | nudix hydrolase 10 | Cytoplasm | enzyme | |
| 65 | NUPR1 | nuclear protein 1, | Nucleus | transcription | |
| | | transcriptional regulator | | regulator | |
| 66 | OTOF | otoferlin | Plasma | other | |
| | | | Membrane | | |
| 67 | PCLAF | PCNA clamp associated | Nucleus | other | |
| | | factor | | | |
| 68 | PKDCC | protein kinase domain | Cytoplasm | kinase | |
| | | containing, cytoplasmic | | | |
| 69 | PLA2G2D | phospholipase A2 group | Extracellular | enzyme | |
| | | IID | Space | | |
| 70 | PPA2 | inorganic pyrophosphatase 2 | Cytoplasm | enzyme | |
| 71 | PPP4R3C | protein phosphatase 4 | Other | other | |
| | | regulatory subunit 3C | | | |
| 72 | PRPH | peripherin | Plasma | other | |
| | | | Membrane | | |
| 73 | PRUNE2 | prune homolog 2 with BCH | Cytoplasm | other | |
| | | domain | | | |
| 74 | RASL12 | RAS like family 12 | Other | enzyme | |
| 75 | RIMS2 | regulating synaptic | Plasma | other | |
| | | membrane exocytosis 2 | Membrane | | |
| 76 | RNASE1 | ribonuclease A family | Extracellular | enzyme | |
| | | member 1, pancreatic | Space | | |
| 77 | ROR1 | receptor tyrosine kinase | Plasma | kinase | cirmtuzumab, |
| | | like orphan receptor 1 | Membrane | | VLS-101 |
| 78 | RPL36AP41 | ribosomal protein L36a | Other | other | |
| | | pseudogene 41 | | | |
| 79 | RRM2 | ribonucleotide reductase | Nucleus | enzyme | docetaxel/ |
| | | regulatory subunit M2 | | | gemcitabine |
| 80 | SCGB2A2 | secretoglobin family 2A | Extracellular | other | |
| | | member 2 | Space | | |
| 81 | SELENOP | selenoprotein P | Extracellular | other | |
| | | | Space | | |
| 82 | SH2D2A | SH2 domain containing 2A | Cytoplasm | other | |
| 83 | SHC3 | SHC adaptor protein 3 | Cytoplasm | kinase | |
| 84 | SLC16A3 | solute carrier family 16 | Plasma | transporter | |
| | | member 3 | Membrane | | |
| 85 | SPATA13 | spermatogenesis | Plasma | other | |
| | | associated 13 | Membrane | | |
| 86 | SPC24 | SPC24 component of | Cytoplasm | other | |
| | | NDC80 kinetochore | | | |
| | | complex | | | |
| 87 | SPP1 | secreted phosphoprotein 1 | Extracellular | cytokine | |
| | | | Space | | |
| 88 | STC1 | stanniocalcin 1 | Extracellular | kinase | |
| | | | Space | | |
| 89 | STC2 | stanniocalcin 2 | Extracellular | other | |
| | | | Space | | |
| 90 | STOML3 | stomatin like 3 | Plasma | other | |
| | | | Membrane | | |
| 91 | SYT6 | synaptotagmin 6 | Cytoplasm | transporter | |
| 92 | TDRD15 | tudor domain containing | Other | other | |
| | | 15 | | | |
| 93 | TEAD2 | TEA domain transcription | Nucleus | transcription | |
| | | factor 2 | | regulator | |
| 94 | TK1 | thymidine kinase 1 | Cytoplasm | kinase | |
| 95 | TM4SF19 | transmembrane 4 L six | Other | other | |
| | | family member 19 | | | |
| 96 | TMEM171 | transmembrane protein | Other | other | |
| | | 171 | | | |
| 97 | TRAF3IP2 | TRAF3 interacting protein 2 | Cytoplasm | other | |
| 98 | TREM2 | triggering receptor | Membrane | transmembrane | anti-TREM2 |
| | | expressed on myeloid cells 2 | Plasma | receptor | antibody |
| 99 | TRPC4 | transient receptor | Plasma | ion channel | |
| | | potential cation channel | Membrane | | |
| | | subfamily C member 4 | | | |
| 100 | TSHZ3 | teashirt zinc finger | Nucleus | transcription | |
| | | homeobox 3 | | regulator | |
| 101 | TUBA8 | tubulin alpha 8 | Cytoplasm | other | docetaxel/ |
| | | | | | gemcitabine |
| 102 | TYMS | thymidylate synthetase | Nucleus | enzyme | pemetrexed, |
| | | | | | capecitabine/ |
| 103 | UBE2C | ubiquitin conjugating | Cytoplasm | enzyme | temozolomide |
| | | enzyme E2 C | | | |
| 104 | UNC80 | unc-80 homolog, NALCN | Plasma | enzyme | |
| | | channel complex subunit | Membrane | | |
| 105 | ZNF219 | zinc finger protein 219 | Nucleus | transcription | |
| | | | | regulator | |
| 106 | ZNF462 | zinc finger protein 462 | Nucleus | transcription | |
| | | | | regulator | |
| 107 | ZNF610 | zinc finger protein 610 | Nucleus | transcription | |
| | | | | regulator | |
| 108 | ZNF880 | zinc finger protein 880 | Other | other | |

TABLE 2
TREM2^{HI} (CLUSTER 12) MACROPHAGE FEATURE GENES.

| Symbol | Entrez Gene Name |
|---|---|
| TREM2 | Triggering Receptor |
| | Expressed On Myeloid |
| | Cells 2 |
| SPP1 | secreted phosphoprotein |
| | 1 |
| RNASE1 | ribonuclease A family |
| | member 1, pancreatic |
| MT1G | metallothionein 1G |
| FOLR2 | folate receptor beta |
| NUPR1 | nuclear protein 1, |
| | transcriptional regulator |
| KLHDC8B | kelch domain containing |
| | 8B |
| CCL18 | C-C motif chemokine |
| | ligand 18 |
| MMP12 | matrix metallopeptidase |
| | 12 |
| APOC2 | apolipoprotein C2 |

TABLE 3
γδ T CELL (CLUSTER 21) FEATURE GENES

| Symbol | Entrez Gene Name |
|---|---|
| RRM2 | Ribonucleotide |
| | Reductase Regulatory |
| | Subunit M2 |
| BIRC5 | Baculoviral IAP Repeat |
| | Containing 5 |
| SPC24 | SPC24 Component Of |
| | NDC80 Kinetochore |
| | Complex |
| UBE2C | Ubiquitin Conjugating |
| | Enzyme E2 C |
| CDCA5 | Cell Division Cycle |
| | Associated 5 |

We claim:

1. A method of determining susceptibility and response to an immune checkpoint therapy in a subject in need thereof, the method comprising:
(a) detecting the expression level of one or more genes associated with an immune cell gene expression signature (ImmuneCells.Sig) of Table 1; and
(b) comparing the expression levels detected in (a) to control expression levels.

2. The method of claim 1, wherein the subject has a cancer selected from basal cell carcinoma (BCC) and melanoma.

3. The method of claim 1, wherein the method comprises treating the subject with an immune checkpoint therapy if the expression level of one or more genes is lower than the expression levels of the control expression levels.

4. The method of claim 3, wherein the one or more genes is associated with macrophages that overexpress TREM2 or a subset of γδ T cells.

5. The method of claim 3,
wherein the immune checkpoint therapy comprises at least one immune checkpoint inhibitor.

6. (canceled)

7. The method of claim 5, wherein the immune checkpoint inhibitor is a PD-1 inhibitor, a PD-L1 inhibitor, a CTLA4 inhibitor, or any combination thereof.

8-10. (canceled)

11. A method for processing a test sample to determine a likelihood that a cancer is responsive to anti-PD-1 immunotherapy in a patient, comprising:
(a) receiving information indicative of an expression level of a plurality of biomarkers in a tumor sample extracted from the patient;
(b) providing the plurality of biomarker levels as input to a classifier configured to predict likelihood that a patient is reactive in response to anti-PD-1 immunotherapy in a computer to classify the test sample, wherein the classifier was trained with a plurality of training samples comprising pre-therapy tumor expression data of known PD-1 therapy responding patients and pre-therapy tumor expression data of known non-responder patients, and wherein the sensitivity and specificity of the classifier is sufficient to identify the likelihood that the patient is responsive to anti-PD-1 immunotherapy;
(c) receiving, from the classifier, an output report that identifies said classification as indicative of the likelihood that the patient is responsive to anti-PD-1 immunotherapy.

12. The method of claim 11, further comprising:
determining, based on the output, that the patient is likely responsive to anti-PD-1 immunotherapy; and
administering anti-PD-1 immunotherapy to the patient based on the determination that the patient is likely to respond to anti-PD-1 immunotherapy.

13. The method of claim 11, wherein the classifier has an accuracy of at least 85%.

14. The method of claim 11, wherein the method comprises:
detecting the expression level of the plurality of biomarkers by sequencing the nucleic acid molecules from the sample to yield data comprising one or more levels of gene expression producing is the sample.

15. The method of claim 14, wherein the method comprises RNA-seq analysis.

16. (canceled)

17. The method of claim 11, wherein the plurality of biomarkers consists of the 10 biomarkers in Table 2.

18. The method of claim 11, wherein the patient's tumor is of a type selected from the group consisting of melanoma and basal cell carcinoma (BCC).

19. The method of claim 11, wherein step (b) comprises identifying a copy number variation or a variant in the nucleotide data.

20. The method of claim 11, wherein said known samples comprise a cancer tissue sample from melanoma or basal cell carcinoma (BCC), and wherein said plurality of training samples further comprises a normal tissue sample.

21. (canceled)

22. The method of claim 11, wherein said sensitivity of at least 70%, and/or wherein said classifier generates said classification at a specificity of at least about 90%.

23. (canceled)

24. The method of claim 11, wherein the test sample is from a patient that was sensitive to checkpoint inhibitor therapy, preferably anti-PD-1 therapy, and wherein the classifier classifies the sample as likely to be responsive to the checkpoint inhibitor therapy.

25. The method of claim 11, wherein the test sample is from a patient treated with anti-PD therapy that was not responsive to checkpoint therapy, and wherein the classifier classifies the test sample as not likely to be responsive to checkpoint therapy.

26. The method of claim 11, further comprising providing a treatment to the subject.

27-28. (canceled)

29. A composition comprising a plurality of nucleic acid probes, wherein the nucleic acid probes of the plurality hybridizes to an mRNA produced by the genes of Table 1.
